SEQUENCE LISTING (46 sequences)

SEQ ID NO: 1 - DNA, 696 bp, Zea mays
  CDS (1)...(693): ZmCBF2
  misc_feature (106)...(147): CBF-specific domain
  misc_feature (154)...(342): AP2 domain
atgtgcccaaccaagaaggggatgaccggagagccgagctcgccatgcagctcggcatcagcctcgaccttaccggagcaccaccagacggtgtggacgtcgccgccgaagcggccagcggggcggaccaagttccgggagacgcggcacccggtgttccgcggcgtccggcgccggggcagcgccgggcggtgggtgtgcgaggtgcgcgtgccggggaggcgcggctgcaggctctggctcggcaccttcgacacggccgaggcggcggcccgcgcgcacgacgccgccatgctcgccctcgccggcgcgggcgcctgctgcctcaacttcgccgactcggcctggctcctcgcggtcccggcctcgtgcgccagcctcgccgaggtccgccacgcggtcgcggacgccgtggaggacttcctccgccatcaggtggtcccggaggacgacgccctcgcggccacgccgtcgtcgccttccagcgaagacggcagcacctctgatggcggggagtcctcctctgattcctctccgcccaccggggcctcgccgttcgaattggatgtgttcaacgacatgagctgggacctgcactacgcgagcttggcgcagggattgctcgtggagccaccgtccgcggtcacggcgctcatggacgaaggcttcgccgatgtgccgctctggagctactag

SEQ ID NO: 2 - protein, 231 aa, Zea mays
MCPTKKGMTGEPSSPCSSASASTLPEHHQTVWTSPPKRPAGRTKFRETRHPVFRGVRRRGSAGRWVCEVRVPGRRGCRLWLGTFDTAEAAARAHDAAMLALAGAGACCLNFADSAWLLAVPASCASLAEVRHAVADAVEDFLRHQVVPEDDALAATPSSPSSEDGSTSDGGESSSDSSPPTGASPFELDVFNDMSWDLHYASLAQGLLVEPPSAVTALMDEGFADVPLWSY

SEQ ID NO: 3 - DNA, 699 bp, Zea mays
  CDS (1)...(696): ZmCBF1
  misc_feature (124)...(165): CBF-specific domain
  misc_feature (172)...(354): AP2 domain
atggagtacgccgccgtcggctacggctacgggtacgggtacgacgagcgccaggagccggcggagtccgcggacggcggcggcggcggcgacgacgagtacgcgacggtgctgtcggcgccacccaagcggccggcggggcggaccaagttccgggagacgcggcaccccgtgtaccgcggcgtgcggcggcgcgggcccgcggggcgctgggtgtgcgaggtccgcgagcccaacaagaagtcgcgcatctggctcggcaccttcgccacccccgaggccgccgcgcgcgcgcacgacgtggccgcgctggccctgcggggccgcgccgcgtgcctcaacttcgccgactcggcgcgcctgctccaggtcgaccccgccacgctcgccacccccgacgacatccgccgcgccgccatccagctcgccgacgccgcctcgcagcaggatgagactgccgccgttgccgctgacgtggtcgcgccctcgcaggcggacgacgtcgccgccgccgccgccgccgcggcggcgatgtacggcggcggcatggagttcgaccactcgtattgctacgacgacgggatggtgagcgggagcagcgactgctggcaaagcggcgccggcgccggtggatggcatagcatcgtggacggcgactacgacgacggcgccagcgacatgacgctctggagctactga

SEQ ID NO: 4 - protein, 232 aa, Zea mays
MEYAAVGYGYGYGYDERQEPAESADGGGGGDDEYATVLSAPPKRPAGRTKFRETRHPVYRGVRRRGPAGRWVCEVREPNKKSRIWLGTFATPEAAARAHDVAALALRGRAACLNFADSARLLQVDPATLATPDDIRRAAIQLADAASQQDETAAVAADVVAPSQADDVAAAAAAAAAMYGGGMEFDHSYCYDDGMVSGSSDCWQSGAGAGGWHSIVDGDYDDGASDMTLWSY

SEQ ID NO: 5 - DNA, 615 bp, Zea mays
  promoter (1)...(615): rab17
  misc_binding (233)...(238): CRT/DRE
  misc_binding (322)...(327): CRT/DRE
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccatggacgccgcc

SEQ ID NO: 6 - DNA, 1625 bp, Arabidopsis
  promoter (1)...(1625): rd29A
  misc_binding (1323)...(1328): CRT/DRE
agcttggttgctatggtagggactatggggttttcggattccggtggaagtgagtggggaggcagtggcggaggtaagggagttcaagattctggaactgaagatttggggttttgcttttgaatgtttgcgtttttgtatgatgcctctgtttgtgaactttgatgtattttatctttgtgtgaaaaagagattgggttaataaaatatttgcttttttggataagaaactcttttagcggcccattaataaaggttacaaatgcaaaatcatgttagcgtcagatatttaattattcgaagatgattgtgatagatttaaaattatcctagtcaaaaagaaagagtaggttgagcagaaacagtgacatctgttgtttgtaccatacaaattagtttagattattggttaacatgttaaatggctatgcatgtgacatttagaccttatcggaattaatttgtagaattattaattaagatgttgattagttcaaacaaaaattttatattaaaaaatgtaaacgaatattttgtatgttcagtgaaagtaaaacaaattaaattaacaagaaacttatagaagaaaatttttactatttaagagaaagaaaaaaatctatcatttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttccttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagttttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggac

SEQ ID NO: 7 - DNA, 1824 bp, Zea mays
  promoter (1)...(1824): RIP2
  misc_binding (1653)...(1658): CRT/DRE
taatcattacttaggttttattttaccacatttttattttgttttcccctgttccttttctcttcatttccattcaattaatgggatgtttgataccttacgattgcaccaacctgttcaattgtacttcagatatcatcttctttgattgtctctcacctctgctttgcttcagtacctgtattttttcccatgaccctgaattctatttgcccacatcacaacacttgcttcttctcgaacaaataaataaacaaacttcacagaaccgtagtttttatttctatccatacattgtcagtttgatgatccagacgaggtagatgaagagaaagaagttgagtatgaagaaatcgaggaggaggttgagtatgaagagatagaggaggattaagaaattgatggtgtgtgtgaatttgatgctaatgatgaaagtaaaatggtcgatgttgatgcgaatgatgagaatgaaaaacggaagcatgctgagcttcttgctcttactcatggagctgaagtttatgttggggcatatcttctaatgtatcttctgaaaatctcaaacaactattctgaagatctcaaacaactatttgaatctgttgggagctgaagtttatgttgggcatatcttctgatgtatcttctctactttagcttttgcatttctattctctgcaaatttagagtccctttttctgcaggttgtatatccttattgtgtcgcatgttttggccgatgctacccgaattgggcaacaatgatctcagaatgtcatgacacacatttgacattgtccatctactattgatcgtgcctgcaagattgaacagatcaagctttgaaagaaggatgtcaaaaggcattggtgattgaacaaaggcagtcaagagccattgaaagaaagttgtatgttgagagcactaagacaacggtcttacagtgtacaaaatatatcactgaatagttatatcttacttttttagcacttgagcaattaaacttttagttgttcattgttatagtcgatacccagatatcatacagtgtctaatatgaacatttaattttcatgtaatcattatgctctaacattttttaaaaaataatgtgctgttgcaacgcacgggcatcgtactagtaaagtatatatatatatatatatatatagacttttaccattcaaaaaaatttgagggcctcaattttttgtttcgccccgggtccatgaaacctagggaccggccgtgtatatatatggtcttcccttcactaactatatagagacagatcacatcggaataaaagaaatttatagaccaaatcggaaacctaaaaaccaaaaaccgagcaattcggtctattcggttttagttagcaggttcaaaatgtccggtcctactaatactcaacaatgattaagaaccgatctgccatattttaaaaaattatggaccggaataacacatagtgaaaagtttaaggagcgaaaatatttttttttccttggcaatttggacggcacgcggagactggcagaccgcatcctcgtgaagcacgttgtccatgcctgaagagagtattctgtattcgcagtattcctgcatttaaaagtttggtgagcgaatcaataattggcataaataatgctaccgacgcatcaccacatagtacgtaccatagtcatccttatcctatcgaattacctacatgcccaaccctcccactacatatatctgcaacgagcgcatcgccaattcacaatgccaattgccagcaacccatccatactttcagctgttgatacaaaaagagaagagaga

SEQ ID NO: 8 - DNA, 1099 bp, Zea mays
  promoter (1)...(1099): RIP2
  misc_binding (930)...(935): CRT/DRE
gcccttgttttggccgatgctacccgaattgggcaacaatgatctcagaatgtcatgacacacatttgacattgtccatctactattgatcgtgcctgcaagattgaacagatcaagctttgaaagaaggatgtcaaaaggcattggtgattgaacaaaggcagtcaagagccattgaaagaaagttgtatgttgagagcactaagacaacggtcttacagtgtacaaaatatatcactgaatagttatatcttacttttttagcacttgagcaattaaacttttagttgttcattgttatagtcgatacccagatatcatacagtgtctaatatgaacatttaattttcatgtaatcattatgctctaacattttttaaaaaataatgtgctgttgcaacgcacgggcatcgtactagtaaagtatatatatatatatatatatatagacttttaccattcaaaaaaatttgagggcctcaattttttgtttcgccccgggtccatgaaacctagggaccggccgtgtatatatatggtcttcccttcactaactatatagagacagatcacatcggaataaaagaaatttatagaccaaatcggaaacctaaaaaccaaaaaccgagcaattcggtctattcggttttagttagcaggttcaaaatgtccggtcctactaatactcaacaatgattaagaaccgatctgccatattttaaaaaattatggaccggaataacacatagtgaaaagtttaaggagcgaaaatatttttttttccttggcaatttggacggcacgcggagactggcagaccgcatcctcgtgaagcacgttgtccatgcctgaagagagtattctgtattcgcagtattcctgcatttaaaagtttggtgagcgaatcaataattggcataaataatgctaccgacgcatcaccacatagtacgtaccatagtcatccttatcctatcgaattacctacatgcccaaccctcccactacatatatctgcaacgagcgcatcgccaattcacaatgccaattgccagcaacccatccatactttcagctgttgatacaaaaagagaagaga

SEQ ID NO: 9 - DNA, 1446 bp, Zea mays
  promoter (1)...(1443): mLIP15
  misc_binding (706)...(710): CRT/DRE
  misc_binding (1426)...(1430): CRT/DRE
  misc_feature 1366, 1374: n = A,T,C or G
  misc_feature 1366, 1374: n = A,T,C or G
  misc_feature 1366, 1374: n = A,T,C or G
aagcttggtaccgagctcggatccactagtaacggccgccagtgtgctggaattcgcccttctgggcaagctgtcactaggactggacaaaatactcgtggctcgataactcgctcgactcgtctcgttagtagctcagctcgactcggctcgttttaattttgtagcgagccaagctagcattctagctcgattctctaatgagccagctcgggttagctcgtgagctagctcgcgagccaaacgagctaagccacaacacaaatttgtctagtcattgatgtcgtctcatctctcatagtcttgttttctcgtagttatgatctgtgatatggacatgtgtggatgtgccatgtacttaaatatttatattattgcatggctacatgtttgtagtgttaaatacttaaaatataatttttcggttataaatatatttatgtacatagatatttatatttagttgtgtggctcacgagcctaacgagctggctcgagcttcctaacgagccgagccgagccagctgtttagcccgttagtataacgagccgagccgagctggctcgttatagtaacgagtcataacgagccgagccataacgagccaagctggttcgatatccacccctagctgtcaccgtcgcccagtccgcttcgttcggtcagcgggccccgcctcatctgcattcttccattctcgtcctccgacctcatctgcattttcccagccaagtagtaggtaaactagtggcggtcccgtggccgtggcatcaggaaaagaatatgccgtcccagcccaccatccccccaccgtcccgaaattcaagagttaccttgggttcaagttataataggctgcccccggtagacgttggaaactttccccttctcgggataaaagataaggagtgtgtgtcctttttttaggataagtccgtgccccttctgtttttcttacattcaggtcttcgcagctcctctattttttgttgtttctttctttcgatctgcgagccgtgcaggtccagtactctcctttctgtgaaggaactcttgcagccggcccctctggtttcgtcgaattcttgttccccggtccctcctcctgtccccgcgtagatccgtccgtccgaggagcacaccgtccccacccccatgtttacccaccagttcctctgacggccgccgtgctccgatgaagatgagcgtgctccgtatccgccgctcccactccttctccgtcgccttcctctactggttctacgtcttctcatgaacgcatcgcccctctccacctgctgatccttcgccgtctctctctctctctctctctctctctcttagatagtcttttgaatccatctntagggctnttgtttctccccatcctccccccaccccaccccccaccaaacagattcaatccgacaagacaagcatccatg

SEQ ID NO: 10 - DNA, 781 bp, Secale cereale
catggacgccgccgacgccggctccccccgttttgggcacaggacggtgtgctcggagccgcccaagaggccggcagggcggaccaagtttaaggagacccgccacccgctgtaccgcggcgtgcggcggcggggtcggctcgggcagtgggtgtgcgaggtgcgcgtgcgcggcgcgcaagggtacaggctctggctcggcacattcaccaccgccgagatggcggcgcgcgcgcacgactccgccgtgctcgcgctcctcgaccgcgccgcttgcctcaacttcgccgactccgcctggcggatgctgcccgtcctcgcggcaggctcgtcccgcttcagcagcgcgcgggaaatcaaggacgccgtcgccgtcgccgtcgtggagttccagcggcagcgccccttcgtgtccacgtcggagacggccgacggcgagaaggacgtccaaggctcgccgaggccgagcgagctgtccacgtccagcgacttgttggacgagcactggtttagcggcatggacgccggctcttactacgcgagcttggcgcaggggatgctcatggagccgccggccgccagagcgtggagcgaggatggcggcgaatacagcggcgtccacacgccgctttggaactagtactagtacacttatccgactaattaagccatgtacagttttagaaactagactactagtggttgtgttcttccaaatatgggaagatacagagtaagcataaggagcaattttcccccgtaaaaaaaaaaaaaaaagggc

SEQ ID NO: 11 - DNA, 902 bp, Arabidopsis thaliana
  CDS (113)...(763)
ctagaacagaaagagagagaaactattatttcagcaaaccataccaacaaaaaagacagagatcttttagttaccttatccagtttcttgaaacagagtactcttctgatcaatgaactcattttctgctttttctgaaatgtttggctccgattacgagtcttcggtttcctcaggcggtgattatattccgacgcttgcgagcagctgccccaagaaaccggcgggtcgtaagaagtttcgtgagactcgtcacccaatatacagaggagttcgtcggagaaactccggtaagtgggtttgtgaggttagagaaccaaacaagaaaacaaggatttggctcggaacatttcaaaccgctgagatggcagctcgagctcacgacgttgccgctttagcccttcgtggccgatcagcctgtctcaatttcgctgactcggcttggagactccgaatcccggaatcaacttgcgctaaggacatccaaaaggcggcggctgaagctgcgttggcgtttcaggatgagatgtgtgatgcgacgacggatcatggcttcgacatggaggagacgttggtggaggctatttacacggcggaacagagcgaaaatgcgttttatatgcacgatgaggcgatgtttgagatgccgagtttgttggctaatatggcagaagggatgcttttgccgcttccgtccgtacagtggaatcataatcatgaagtcgacggcgatgatgacgacgtatcgttatggagttattaaaactcagattattatttccatttttagtacgatactttttattttattattatttttagatccttttttagaatggaatcttcattatgtttgtaaaactgagaaacgagtgtaaattaaattgattcagtttcagtat

SEQ ID NO: 12 - protein, 216 aa, Arabidopsis thaliana
MNSFSAFSEMFGSDYESSVSSGGDYIPTLASSCPKKPAGRKKFRETRHPIYRGVRRRNSGKWVCEVREPNKKTRIWLGTFQTAEMAARAHDVAALALRGRSACLNFADSAWRLRIPESTCAKDIQKAAAEAALAFQDEMCDATTDHGFDMEETLVEAIYTAEQSENAFYMHDEAMFEMPSLLANMAEGMLLPLPSVQWNHNHEVDGDDDDVSLWSY

SEQ ID NO: 13 - protein, 245 aa, Zea mays
  PEPTIDE (1)...(245): ZmCBF3
  DOMAIN (28)...(41): CBF-specific
  DOMAIN (44)...(105): AP2
MDADDSSYASSSSFSPPPSPADHLRLPPKRRAGRKKFRETRHPVYRGVRARAGGTRWVCEVREPQAQARIWLGTYPTPEMAARAHDVAAIALRGATAADLNFPDSAHALPRARTAAPDDIRRAAAQAAELYRPSPSSSSASGLLLHHGRRTIAAPPPPLSLPPPEASSTCCWTTSTGTRGAGTTPTCCDGFLDEDSIFDMPGLIHDMAWGMLLTPPAMGRGLDWGALDDDDDHSHVDCTLWTLDG

SEQ ID NO: 14 - protein, 222 aa, Zea mays
  PEPTIDE (1)...(222): ZmCBF4
MPPPPSSSSSPSQDAGSPKRAAGRNKFRETRHPVFRGVRRRGRAGGRWRWVCEVRVPGRRGCRLWLGTFAAAEAAARAHDAAMLALRGGAARARCLNFPDSAWLLDVPVLPLPHGAAPWADVRRAVAIAVEGFFRARPAAEDAMSATSEPSSAATEAEAEASSSSGTDGGAPEASPFELDMLSDMGAGLYYACLAQGLLVEPPPLDAPCPDDSDCGLALWSY

SEQ ID NO: 15 - protein, 264 aa, Zea mays
  PEPTIDE (1)...(264): ZmCBF5
MDTAGLVQHATSSSSTSTSASSSSSSSEQQSKAAWPPSPASSPQQPPKKRPAGRTKFRETRHPVFRGVRRRGAAGRWVCEVRVPGRRGARLWLGTYLAAEAAARAHDAAMLALQGRGAGRLNFPDSARLLAVPPPSALPGLDDARRAALEAVAEFQRRSGAADEATSGASPPSSSPSLPDVSAAGSPAAALEHVPVKADEAVALDLDGDVFEPDWFGDMDLELDAYYASLAEGLLVEPPPPAAAWDHEDCCDSGAADVALWSYY

SEQ ID NO: 16 - protein, 231 aa, Zea mays
  PEPTIDE (1)...(231): ZmCBF6
MDAAGSFSDYSSGTPSPVAGGGGGDDFGSSSYMTVSSAPPKRRAGRTKFKETRHPVYKGVRRRNPGRWVCEVREPHGKQRIWLGTFETAEMAARAHDVAALALRGRAACLNFADSPRLLRVPPTGSGHDEIRRAAAVAADQFRPAPDQGNVAAEEEAADTPPPDALPSVTMQSVDDDPYCIIDDRLDFGMQGYLDMAQGMLIDPPPMAGSSTSGGGGDDDDDDGEVKLWSY

SEQ ID NO: 17 - protein, 246 aa, Zea mays
  PEPTIDE (1)...(246): ZmCBF7
MDMGRHQLQLQHAASSSSTSASSSSEQDKPLCCSGPKKRPAGRTKFRETRHPVFRGVRRRGAAGRWVCEVRVPGRRGARLWLGTYLGAEAAARAHDAAMLALGRGAACLNFPDSAWLLAVPPPPALSGGLDGARRAALEAVAEFQRRRFGAAAADEATSGTSPPSSSSSATKPAPAIERVPVEASETVALDGAVFEPDWFGDMDLDLYYASLAEGLLVEPPPPPPPAAWDHGDCCDSGADVALWSY

SEQ ID NO: 18 - protein, 238 aa, Zea mays
  PEPTIDE (1)...(238): ZmCBF8
  VARIANT 176: Xaa = Any Amino Acid
  VARIANT 176: Xaa = Any Amino Acid
MCPTKKEMSAESSGSASSWTSASASASTSTSPEHQTVWTSPPKRPAGRTKFRETRHPVFRGVRRRGSAGRWVCEVRVPGRRGCRLWLGTFDAAEAAARAHDAAMLAIAGASACLNFADSAWLLAVPASYASLADVRRAVAEAVEDFQRREAAAGDDARSATSPTPSTSGTDDDAAXDGEESSPATEVSSFQLDVFDNMSWDLYYASMAQGMLMELPSAVPAFGDDGYTNVADVPLWSY

SEQ ID NO: 19 - protein, 227 aa, Zea mays
  PEPTIDE (1)...(227): ZmCBF9
MDSTDPPPASPSSPGPPGGQPSPPKRPAGRTKFQETRHPVFRGVRRRGRAGRWVCEVRVPGSRGDRLWVGTFDTAEAAARAHDAAMLALCGAAASLNFADSAWLLHVPRAPAGLPGVQRAATDAVAAFLRTQQPRGGGDAPAAASQGQRANAATETELDHAGASAAAVDGGGGSVVEVDVFGGMDDAGSYYASLAQGLLIDPPPPAVECPEEEDDDCGGAGEMELWD

SEQ ID NO: 20 - protein, 230 aa, Zea mays
  PEPTIDE (1)...(230): ZmCBF10
MCPIKRETTSAESGSPCSSSTSTSSSSEHHQTAWASPPKKRPAGRTKFRETRHPVFRGVRRRGRAGRWVCEVRVPGRRGCRLWLGTFDTAEAAARAHDAAMLAVAAGAARLNFADSAWLLAVPTASYASLADVRRAVAEAVESFLRRREEEEGDALSAASSTSPNDKDGDESSSATTTDDDSPFELDMFGGMSWDLYYANLAQAMLVEPPPIVPALCDDGVASELPLWSY

SEQ ID NO: 21 - protein, 222 aa, Zea mays
  PEPTIDE (1)...(222): ZmCBF11
MDWAYYGSGYSTPASGGGGGDEDAYMTVSSAPPKRRAGRTKFKETRHPVYKGVRSRNPGRWVCEVREPHGRQRIWLGTFETAEMAARAHDVAALALRGRAACLNFADSPRRLRVPAQGAGHDEIRRAAVEAAELFRPQPGERNVGGSEAAAEAAAAAPCAMGSGDLGGGEFPYYPVDDGLEFEMRGYLDMAQGMLIDPPQPAAGQSAWIEDEYECEVSLWSY

SEQ ID NO: 22 - protein, 340 aa, Zea mays
  PEPTIDE (1)...(340): ZmCBF12
MAAAIDMYKYYNTSAHQIASSSSPSDQELAKALEPFITSASSSSSSSPYHGYSSSPSMSQDSYMPTPSYTSYATSPLPTPAAASSQLPPLYSSPYAAPCMTGQMGLNQLGPAQIQQIQAQFMFQQQQQQRGLHAAFLGPRAQPMKQSGSPPPLAPAPAQSKLYRGVRQRHWGKWVAEIRLPKNRTRLWLGTFDTAEDAALAYDKAAFRLRGDTARLNFPALRRGGAHLAGPLHASVDAKLTAICQSLSESKSKSGSSGDESAASPPDSPKCSASTTEGEGEEESGSAGSPPPPPPPTLAPPVPVPEMAKLDFTEAPWDETEAFHLRKYPSWEIDWDSILS

SEQ ID NO: 23 - protein, 279 aa, Zea mays
  PEPTIDE (1)...(279): ZmCBF13
MAAAINLPGPSEDLMRAMESFMQDDAPSPLAMPPAPSFPAAAAHGAQYPATHLSPAQMQFIQAQLHLQRNPGLGPRAQPMKPAVPVPPAPAPQRPVKLYRGVRQRHWGKWVAEIRLPRNRTRLWLGTFDTAEQAALAYDQAAYRLRGDAARLNFPDNAESRAPLDPAVDAKLQAICATIAAASSSSKNSKAKSKAMPINASVLEAAAASPSNSSSDEGSGSGFGSDDEMSSSSPTPVVAPPVADMGQLDFSEVPWDEDESFVLRKYPSYEIDWDALLSN

SEQ ID NO: 24 - protein, 297 aa, Zea mays
  PEPTIDE (1)...(297): ZmCBF14
MAATIDLSGEELMRALEPFIRDASAHGSSPLLHPHQPLSPSSPFSFHQAVAAASSYGGNYPFAAADEAGQLSPSQMQYIQARLHLQRRQAQTSVLGPRAQPMKASASAAPAPARPQKLYRGVRQRHWGKWVAEIRLPRNRTRLWLGTFDTAEEAALAYDQAAYRLRGDAARLNFPDNAASRGPLHASVDAKLQSLCQSIAASKKGAKKPASAAAAASSSAPTSNCCSSPSSDDATSSCLESATESSCPSPSPSASPGPTVPEMQQLDFSEAPWDEAAGFALTKYPSYEIDWDSLLAN

SEQ ID NO: 25 - protein, 244 aa, Zea mays
  PEPTIDE (1)...(244): ZmCBF15
MAMDSSSSGSEPTSSSSAEAPASPTASSSESSAAGSKKRRRSKDGHHPTYRGVRMRAWGKWVSEIREPRKKSRIWLGTFPTAEMAARAHDAAALAIKGRATQLNFPVLAGVLPRAASAAPKDVQAAAMLAAAFTSPSSAPSELDAGAPAPREVPATKNGSPSEDEAGAEAPVPPAAASQPGTPSSGVDEERQLFDLPDLLLDVRDGFGALPADVGPVPRVGGQCGGGAAALGIAVLQLQRGSKQ

SEQ ID NO: 26 - protein, 281 aa, Zea mays
  PEPTIDE (1)...(281): ZmCBF16
MAQELHETSSCSATTTSSCTTSCCSSTVTDSSSSPPSPAAANAAPATRKRQALEAEAEAEAGGEEEEEEEEGCAGNKAAPAKKRPRGSEGKHPTFRGVRMRAWGKWVSEIREPRKKSRIWLGTFPTAEMAARAHDVAALAIKGRAAHLNFPDLAGALPRAASAAPKDVQAAAALAAAFTSPSSEPGAGAHEEPAAKDGAAPEEAAADAQAPVPVALPPPAASRPGTPSSGVEDERQLFDLPDLLLDIRDGFGRFPPMWAPLTDVEDVVNAELRLEEPLLWE

SEQ ID NO: 27 - protein, 262 aa, Zea mays
  PEPTIDE (1)...(262): ZmCBF17
MVKTAASSSSDDAAAAKRRTYKGVRMRSWGSWVSEVRAPGQKTRIWLGSHATAEAAARAHDAALLCLRGSAADLNFPLRLPFDLPPAATMSPKAIQRVAAAAAAAASGSASSCGGLRAPPFAPPDENSGTSACSDGDATPASSTTSSPTSDSAPAWSTTSHADDVSFPGVHRQQRRRRRLLRRARGHRVLLPVAQVHGVRYDGPMQHVLRASAHGDGRGQRVGVGGGRRDRPLELLVPQLIARHQFGGQAGLKRSPFVSNQR

SEQ ID NO: 28 - protein, 250 aa, Zea mays
  PEPTIDE (1)...(250): ZmCBF18
MKGKGGPDNTQCGYRGVRQRTWGKWVAEIREPNRVDRLWLGTFPTAEDAARAYDEAARAMYGDLARTNFPGQDATTSAQAALASTSAQADPTAVEALQTGTSCESTTTSNYSDIASTSHKPEPEASDISSSLKAKCPAGSCGIQDGTPSVADKEVFGPLEPITNLPDGGDGFDIGEMLRMMESDPHNAGGADAGMGQPWYLDELDSSVLESMLQPEPEPEPEPFLMSEEPDMFLAGFESAGFVEGLERLN

SEQ ID NO: 29 - protein, 316 aa, Zea mays
  PEPTIDE (1)...(316): ZmCBF19
MAAAIDMYKYCNTSAHLIASSSPSDQELAKALEPFITSASSPYHRYSLAPDSYMPTPSSYTTSPLPTPTSSPFSQLPPLYSSPYAASTASGVAGPMGLNQLGPAQIQQIQAQLMFQHQQQRGLHAAFLGPRAQPMKQSGSPPAQSKLYRGVRQRHWGKWVAEIRLPKNRTRLWLGTFDTAEGAALAYDEAAFRLRGDTARLNFPSLRRGGGARLAGPLHASVDAKLTAICQSLAGSKNSSSSDESAASLPDSPKCSASTEGDEDSASAGSPPSPTQAPPVPEMAKLDFTEAPWDETEAFHLRKYPSWEIDWDSILS

SEQ ID NO: 30 - DNA, 738 bp, Zea mays
  CDS (1)...(738)
  misc_feature (82)...(123): CBF-specific domain
  misc_feature (130)...(300): AP2 domain
atggacgccgacgactcctcgtacgcatcgtcctcctcgttctctccgccgccgtcccccgccgatcatctccgcctgcccccgaagcggcgtgcgggccgcaagaagttccgggagacgcggcacccggtgtaccgcggcgtgcgcgcgcgcgccggtggcacccgctgggtgtgcgaggtgcgggagccgcaggcgcaggcgcgcatctggctcggcacctacccgaccccggagatggccgcgcgcgcgcacgacgtcgccgccatcgcgctccgcggcgccaccgccgccgacctcaacttcccggactctgcccacgcgctcccgcgcgcgcgcaccgccgcgcccgacgacatacgacgcgccgccgcgcaggccgccgagctctaccgcccgtctccttcttcctcttccgcctccggcctgctgctgcaccacggccgccggacgatcgctgccccgccaccgccgctctcactgccgccgccggaggcttcttctacctgctgctggacgaccagtacaggaacccgaggagcaggtaccacgccgacctgctgcgacggcttcctggacgaggattccatctttgacatgccagggctcatccacgacatggcctgggggatgctgctcacgccaccagccatgggccggggcctcgactggggcgcccttgacgacgacgacgaccatagccacgtcgactgcacgctctggacgctggacggatga

SEQ ID NO: 31 - DNA, 561 bp, Zea mays
  misc_feature 538, 539, 540, 558, 559, 560, 561: n = A,T,C or G
  misc_feature 538, 539, 540, 558, 559, 560, 561: n = A,T,C or G
ccacgcgtccgccagaaccagctcactcctcactccacttccactcccaacagcaagctcaagcagtcagtcaccggcaggggtcagggtcacagtcacagcagcagccatggacacggccggcctcgtccagcacgcgacctcctcgtcttccacctccacctcggcgtcgtcgtcctcgtcctcgtccgagcagcagagcaaggcggcgtggccgccgtcgcccgcttcctccccgcagcagccgcccaagaagcgccccgcggggcgcacgaagttccgggagacgcggcacccggtgttccgcggcgtgcggcggcggggcgccgcgggccggtgggtgtgcgaggtgcgcgtcccggggaggcgcggcgcgcggctgtggctcggcacctacctcgccgccgaggcggcggcgcgcgcgcacgacgccgcgatgctcgccctgcagggccgcggcgcggggcgcctcaacttcccggactccgcgcggctgctcgccgtgccgcccccgtccgcgctcccgggcctggacgannnccgccgggcggcgctcgnnnn

SEQ ID NO: 32 - DNA, 1430 bp, Zea mays
  misc_feature 928, 931, 934: n = A,T,C or G
  misc_feature 928, 931, 934: n = A,T,C or G
ccaaaaaaaaaatctactttgattttccataaagaaaacctctcagcctctttcttctagctccatccaagctttagacctaatggccgcagccatcgacatgtacaagtactacaataccagcgcacaccagatcgcctcctcctcctccccctcggatcaggagctcgcgaaagcactcgagccttttataacgagtgcttcctcctcttcgtcctcctccccctaccatggctactcgtcctctccatccatgtcccaagattcttacatgcctacaccttcttacaccagctacgccacctcgcctcttcccactcccgccgctgcctcctcgcagcttccgccactctactcgtcgccttatgcggcgccgtgcatgaccggccagatgggcctgaaccagctcggcccggcccagatccagcagatccaggcccagttcatgttccagcagcagcagcagcagaggggcctgcacgcggcgttcctgggcccgcgggcgcagccgatgaagcagtccgggtcgccgccgccgctggcgccggcgccggcgcagtcgaagctgtaccgcggcgtgcggcagcgccactggggcaagtgggtggcggagatccgtctcccgaagaaccgcacgcggctgtggctcggcaccttcgacaccgccgaggacgcggcgctcgcctacgacaaggcggccttccgcctccgcggcgacacggcgcgcctcaacttcccggccctccggcgcggcggcgcgcacctcgccggcccgctgcacgcgtccgtggacgccaagctgaccgccatctgccagtccctgtcggagtccaagtccaagagcggctcgtcgggcgacgagtcggccgcgtccccgccggactcccccaagtgctcggcgtcgacgacggagggggaaggggaggangantcnggctccgccggctcccctcctcctcctcctcccccgacgctggcgccgcccgtgccggtgccggagatggcgaagctggacttcacggaggcgccgtgggacgagacggaggccttccacctgcgcaagtacccgtcctgggagatcgactgggattccatcctgtcatgagcaataatagctccgtgtaatttaattttctactgtctgggttttgcggctgcggtggcccgatggcattttagacgtcggccatggcggctgcaagtagcaatgagtaactagctagctagtacatcgtcgtcgactcgtcgtcgtccagtgttgtgaagcagcgtcaagtacatgcgtgctagtctctcctggttgagctgccggttgtttttttttctcacggcacggccagtcgagaagagtcagtagtgtaatctcgtggtgttatgatcatcggttgcagcttatgtaaaaaaaaaaaaaaaaaaaaaaaaaaaaaaaa

SEQ ID NO: 33 - DNA, 475 bp, Zea mays
  misc_feature 445: n = A,T,C or G
  misc_feature 445: n = A,T,C or G
gaggcttgagcgacccgagcaaaaaaaaatcatccgcgaagcagcgacgcaggtttgttctctgatctctctggttcttcttttagcagcagggacgcacagcacaagaaaaggtccggttcaagaggctcttctgttgggtttttttttttagaatcctcaagagaagagtgttcttgggttaggatttttttctcacctcatcacacctctcctaggaggaacagagatccagagaaccttttttctcttctggatccttgccgccttttggccttcgaaagggggttgttttgcaataccctctctctctgttttttttttttttatcttggcacaaacccacctcgtttctctacttttattgaggaacagagctggttttccaaaaaaaaaatctactttgattttccataaaaaaaacctctcagcctctttcttctanctccatccaagctttaaacctaatggccgc

SEQ ID NO: 34 - DNA, 1241 bp, Zea mays
gtcgtgcctgttctttgattttagctaagaaccagccgatttagtcttcttcttccttacaaaaaaattccccagaaaaaaaaaagttgtgagtagtatattcttgtaccatttgatggccgctgctataaatctccccgggcccagcgaggatctgatgcgagcgatggagtccttcatgcaagacgacgccccatcccctctggccatgccgccggcgccgtctttcccagcggccgcagcccacggggcccagtacccggcgacccacctgagcccggcgcagatgcagttcatccaggcccagctccacctgcagcggaacccggggctgggcccgcgggcgcagcccatgaagcccgccgtcccagtgccgccggcgccggcgccgcagcggcctgtgaagctgtaccgcggcgtgcggcagcgtcactggggcaagtgggtggccgagatccggctcccccggaaccgcacccgcctgtggctcgggaccttcgacaccgccgagcaggcagcgctggcctacgaccaggcggcgtaccgcctccgcggggacgcggcgcggctcaacttccccgacaacgcggagtccagggcgccgctcgaccccgccgtggacgccaagctgcaggccatctgcgccaccatcgccgccgcgtcgtcgtcatccaagaattccaaggccaagagcaaggcgatgccaatcaacgcgtccgttctggaagcggcagcggcgtctccgagcaacagctcctccgacgaaggttccggctccgggttcgggtcggacgacgagatgtcctcgtcctccccgacgccggtggtggcgccgccggtggcggacatgggacagctggatttcagcgaggttccgtgggacgaggacgagagcttcgtgctccgcaagtacccgtcctacgagatcgactgggacgcgctgctctccaactagtcgcccttcgccgacagatgtgctgttgtagttcagtagtggcagtgtctctggccgccgcagatgaggttttaggcaatctgcaggccgccggcccatgtgtattaagtaggttttgctcagttgttggccctggacttcgccggcgtttttgtgaccggcgtccccgagtgcactgcattggtgtactggtctgtctgtaaaaaaaatggatctgtacttctatagtgtgtattcaaccattgttcttagttgtgatgttactcgtgctgcaaaaaaaaaaaaa

SEQ ID NO: 35 - DNA, 776 bp, Zea mays
ccacaatccccttcaacaaacgcaccgcactccacggcagccagaaaacacaatccacacagtctccgtgaggtctgagcgaggtaggactagctctgagcgagttcttccgtcttccaggtataatccttgcaactctcactatcctcttcgcatcgctcgtctctttttgagactagttggatttctcttctttttccttggatcctactacctactagaacagagctccggagccttttagtttctggtatctgatctactcattttcttttcttccccttttggtgacataggtaggtctagttaagatctgtcgtgccagttctttgattttagctaagaaccaggcgatttagtcttcttccttacaaaaaaaaaatccccagaaaaaaaagttgtgagtagtatattcttgtaccatttgatggccgctgctataaatctccccgggcccagcgaggatctgatgcgagcgatggagtccttcatgcaagacgacgccccatccccgctggcctacgaccaggcggcgtaccgcctccgcggggacgcggcgcggctcaacttccccgacaacgcggagtccagggcgccgctcgaccccgccgtggacgccaagctgcaggccatctgcgccaccatcgccgccgcgtcgtcgtcatccaagaattccaaggccaagagcaaggcgacgccaatcaacgcgtccgttctggaagcggcagcggcgtctccgagcaacagctcctccgacgaaggttccggctccgggt

SEQ ID NO: 36 - DNA, 720 bp, Zea mays
aattaacccctcactaaagggaataagcttgcggccgcagaagctgtaccgcggcgtgcggcagcggcactggggcaagtgggtggcggagatccggctcccgcgcaaccgcacccggctctggctcggcaccttcgacaccgccgaggaggcggcgctggcctacgaccaggccgcctaccgcctccgcggcgacgcggcgcgcctcaacttccccgacaacgccgcctcccgcggcccgctccacgcctccgtcgacgccaagctccagagcctgtgccagagcatcgccgcgtccaagaagggcgccaagaagccagcctccgccgcagctgccgcgtcgtcgtccgcccccaccagcaactgctgctcctcgccgtcgtccgacgacgcgacctcgtcctgcctcgagtccgccaccgagtcctcgtgcccgtccccgtccacgtcgccgtccgcctcgcccgggccgacggtgccggagatgcagcagctggacttcagcgaggcgccgtgggacgaggccgccggcttcgcgctcaccaagtacccgtcgtacgagatcgactgggactccctcctcgccaattaatcgtcgccccctgttggctggctggctggctcccttcctacccacactgccgtcgtgcagctggaggagggttaaagaaggttaacggccgtcgcgcagatgggattttagacatcctgcgcgggcac

SEQ ID NO: 37 - DNA, 733 bp, Zea mays
  misc_feature 663, 691: n = A,T,C or G
  misc_feature 663, 691: n = A,T,C or G
tcctctgccaccaccaccacctcgtcgtgtaccacctcttgctgctcgtccactgtcacggactcgtcctcgtcgcccccgtcaccggcggcggccaatgccgcgcccgcgccacggaaggcggaggccgagactggcggggacgaggacggttgtgctggtaaggcggcgccggcgaagaagcgggagcggagcagcgaggggaagcacccgacgtaccgcggcgtgcggatgcgggcgtggggcaagtgggtgtcagagatccgcgagccgcgcaagaagtcgcgcatatggctcggcacgttcccgaccgccgagatggccgcgcgcgcccacgacgccgccgcgctcgccatcaagggccgcgccacgcaactcaactttccggtcctcgccggcgtgctcccgcgcgccgcgtccgcggcgcccaaggacgtccaggccgccgccatgctggccgccgcgttcacgtcgccgtcgtcagcaccatcggagctcgacgccggcgcgccggcgccacgcgaagtgcccgccacaaagaacggctccccgtccgaggacgaggcaggcgctgaggcgccggtaccaccggcggcggcctctcagccagggacgccgtcgagcggcgtggacgaagagcggcagctgttcgacttgccgganctgctcctcgacgtccgggacgggttcngggcgcttcccgccgatgtgggccccgttccatgacgtgttg

SEQ ID NO: 38 - DNA, 509 bp, Zea mays
acttgatcctcctgcacaccagagtaccagacaccactgcgcatcagtgcaaagaggtagctaccctatctgccatggctcaagagctccacgaaacgtcctcttgctctgccaccaccacctcgtcgtgcaccacatcctgctgctcgtccactgtcacagactcgtcctcttcgcccccgtcaccggcggcggccaatgccgcgcccgcgacacggaagcggcaggcgttggaggccgaggccgaggccgaggcgggcggtgaggaggaggaggaggaggaggaaggctgtgctggtaataaggcggcgccggccaagaagcgaccgcggggcagcgaggggaagcacccgacgttccgcggcgtgcggatgcgggcgtggggcaagtgggtgtcggagatccgcgagccgcgcaagaagtcgcgcatatggctcggcacgttccccaccgccgagatggccgcgcgcgcccacgacgtcgcggcgctcgccatcaagggccgcgcc

SEQ ID NO: 39 - DNA, 684 bp, Zea mays
  misc_feature 655, 675, 676, 677, 678, 679: n = A,T,C or G
  misc_feature 655, 675, 676, 677, 678, 679: n = A,T,C or G
gataattcattatacattacttgcacttgcagaaaaacacagcttcgtctacacatagcctggcatgcaacggagaggagattgactccatggaaagctgccttaaaacatgtagctccatgagagcctccctccacattgcataattagcctagcactagtaatatatagtgcaataattaactagtggtatgaatcaaataatccttcatagcatatttatatatgatgcttatttataccgtgaccaattcttgatcacgtaggttggttgcagttgtaattaatcagtcagcgttgatttgagacgaatggtgaccgcttgaggccggcttgacctccgaactgatgtctggcgatcagttgagggacgagaagctccagaggtcgatctcgccttcctcctcccactcccactcgctggccgcgtccgtcgccatgggcgctggcgcgaagaacgtgctgcatgggtccatcatagcgtactccatgcacttgggcgaccggaagaaggactcgatgtccgcgagcgcggcgaggtccgcgtcgcccgccaccatgtccacgtcgtcgtcgtgcgccacgccgtagcacaggtcatcagtgttcccggccgtctccggggagctcacgcccgacgacgcgtcgccgtagntgcacggaggggtggcgccnnnnncggcc

SEQ ID NO: 40 - DNA, 704 bp, Zea mays
  misc_feature 639, 640, 641, 642, 661, 662, 663, 664, 665, 685, 686,688, 692, 694, 695, 696, 697, 704: n = A,T,C or G
  misc_feature 639, 640, 641, 642, 661, 662, 663, 664, 665, 685, 686,688, 692, 694, 695, 696, 697, 704: n = A,T,C or G
ctagacatctcgcgggtcttatcgactccaacaagaacacactacacaccagccagcgagatagcgaacgctaggaacccagtggccatctttggagcggccatgacgctggatcagaaccatgccatgccgatgcagcccccggccctgcagcccggaagagcatatggagcagagggcagtgctgtggtgcatggttccatcagaacagtaggaagaagcgacctcgcagatcacgagatgggcctacgtcagtggcagctgtcatccagcggtgggctgagcgcaacaagcatttggagtatgaggaatctgaggaggcaaagcgaccaagaaaagcacctgcaaagggttcaaagaagggctgtatgaagggaaaaggggggcctgacaatactcaatgtggataccgtggagtgaggcagcgtacttgggggaagtgggttgctgaaataagagagccaaatcgtgtcgacagactctggctgggtaccttcccaaccgcggaggatgcagctagggcctatgatgaggcagccagagcgatgtatggagacttggcacggactaacttccccggacaggatgcaacaacctctgcccaagctgctctatcatcgacctctgcccaggctgctnnnncagctgttgaagctcttcnnnnnggcacgtcatgcgagtcgannangacntnnnntcactcn

SEQ ID NO: 41 - DNA, 722 bp, Zea mays
gccacatcccatcccaactccacggtgaggtttgagcgatccgagcaaaaaaaatcaaccgaggcgcagcaacgcaggttcgttctctgaactctctggttcttttagcagcagggacgcgcagcacaagaaaggtccggttcaagaggctcttcttttgggtttattagaatcctcaaaggaagcgtgttcttgggttaggatttttttttctcacctcatcacacctctcctaggaggagcagagatccagagaaccttttttctcttctggattctcaccgcttcttttttttttctctgcacaaacacaccctcgtttctctacttttattgaggaacagggctgatttccaaactttttcctacccctgttttcataaacaagctccttttatttcttcccgttttaagttctgcccagcctcttcttctagctccatccaagctttctccatctagtagttccaatggccgcagccatagacatgtacaagtactgcaataccagcgcacaccttatcgcctcctcgtccccctcggatcaggagctcgcgaaagcactcgagccttttataacgagtgcttcctccccctaccatcgctactcgttggccccagattcttacatgcctacaccctcctcctacaccacctcgcctcttcccacccccacctcctcgcctttctcgcagcttccgccactctactcgtcgccttac

SEQ ID NO: 42 - DNA, 1949 bp, Secale cereale
  promoter (1)...(1949): Rye CBF31 promoter
ataagcatgaccatgagccatttcgcatcaccttcaagaagggaactcgtgcccaaaggcgtcattgttgcgagtgttgaagcaaggacaaaatttctccttgagaaagagtagagccacatcttatatatgcgaaaacaacacacacacacacacacacacacatggcacgcgacaacatcgacgacagcacataaccaaatggcaacagatgaaagaatggcgtaaccattgaggccagacggggcgcgataaagctatgtcaaacaagggctacatggatcttgtgcggaaccagcagaaggcggcagatcaatgaaagatctatccaacttagatattgtccatccatggcatgagctagtggattctagcgtgggggcctccggaatcgcgagagcgacccaggaggcaggggacacttttcacaaagtttgagttgggggaggagggcagcaagtacttgtaatcacatatattgtgattgattagttactaaacatatgtttgtctcgtttgtctccacgtctagagtagaggcacaatcccaaccaccacctccaaaattctcccacacgccgccgcaacggtccccttccactttctcgtctcgcggcaaaaggagtgagaaccctttgtatgttacctttttttattcttaggttttgttttcctgacgacatcaccaaggcgatggcggtctcttcctacctcaacaacatccgatacagcactaccgaagggcgcgtgtgagtttttgtccctggatgtaatggctctcttcagatcttggtctttgttttgtttttgtccccggatctaccggttctcctcagatattggtcttccatgggcagctattagacaatttagacatgacctgtgggagtatgtgataatttgctagttagtgaaattgatttctaccgacaaaaacataaaaaccattggaaattattggagtgttgatggttatggtatagtccagtttttcttttggagaaagagaggtctgtctagagcacatctatcttagttattgtacatctaagtgactcagtcaaactaaaaagaaaaagaaaaaagattaaaaaatgcttacacgaatcttagcgtaagattaagaacaaagttggaagtacacttttcaaaggacggagggagtagcacttagatgtcaatacttaggacacatctttatgtgttttaggaaaactggagaaaaaagatatgctccttttcaagaattaaagtaagaaaacaacgacgtgcccttaatttgttagtcgatcaagaatcagttcccgtcgctcacgcgtctggaaggccagcgtatgcagccgcaaatccctccccgatatacccaagtactccgtacgatatacaaaaaggttgttctcgcacgattacaacttttgattagataaaaatgatgtgcagctccccgaaaagaaaagtagggaacaaagatatagtgtgctcatccgtatggaatctattatggcgtcgaaacgtctagaagggcgccacagccttcaaagcccttccgagatgaacaatctcggggtgaacaagcagacaccagtgcatctcatggcaaaccagaaaaaatgtaacaaaagtagcaccgtggtggtacgtccaagcgagagttacctcgatgaagctgcctactgctcgctagtgtaagtgagagaaagaagaaccgggattttccattagaaaccaatctgccgtgagagagtccatttccacccgagcgtccacgtcgtggcgggtacccaacccgttgccagtagccccaaactactcacctgcttgattccccgcttctagttctcatcggagctacaatccatcgaccctcactacaacggcttaacgcgcaccacaccccgccccgctacgctgcacactccggtccggtgttatacgcccccccgctacag

SEQ ID NO: 43 - DNA, 669 bp, Zea mays
  CDS (1)...(669): ZmCBF4
atgccgccgccgccgtcctcgtcgtcttcgccctcgcaggacgccggcagccccaagcgagccgcggggcgcaacaagttccgggagacgcggcacccggtgttccgcggcgtgcgccggcgaggccgcgcggggggccggtggcggtgggtgtgcgaggtccgcgttccaggccgccgcggctgcaggctctggctcggcaccttcgcggccgcggaggccgccgcgcgcgcgcacgacgccgccatgctcgcgctccgcggcggcgccgcgcgcgcgcggtgcctcaacttcccggactcggcctggctgctggacgtgccggtgctgccgctgccccacggcgcagcgccctgggccgacgtccgccgcgccgtcgcgatagccgtcgaggggttcttccgggcgcggccagccgccgaggacgccatgtccgccacctcggagccgtcgtcagcggccacggaggcggaggcggaggcgtcctcctcctccgggaccgacggcggcgcgccggaggcctcgccgttcgagctggacatgctgagcgatatgggcgccggcttgtactacgcgtgcttagcgcaggggctgctcgtggagcctccaccgttagacgcgccgtgccccgacgacagcgactgtggcctcgcgctctggtcctactga

SEQ ID NO: 44 - DNA, 696 bp, Zea mays
  CDS (1)...(696): ZmCBF6
atggacgccgccggctccttcagcgactactcctctggaaccccgtcccctgtcgccggcggcggcggcggcgacgacttcggctcctcctcctacatgacagtgtcatcggcgccgcccaagcgccgagccgggcggaccaagttcaaggagacgcggcaccccgtgtacaagggcgtgcggcggaggaaccccgggaggtgggtctgcgaggtgcgggagccgcacggcaagcagcggatatggctcgggaccttcgagaccgccgagatggcggcgcgcgcgcacgacgtcgccgcgctcgcgctgcgcggccgcgccgcctgcctcaacttcgccgactcgccgcggctcctcagggtgcccccgacgggctccgggcacgacgagatacgccgcgcggccgccgtggcggcggaccagttccgcccggcgcccgatcagggcaatgtggccgccgaggaggaggcggccgatacaccaccaccggatgccttgcccagcgtgacgatgcagagcgtcgacgacgacccgtactgcattatcgacgacaggctcgacttcgggatgcaggggtacctcgacatggcgcaagggatgctcattgatccgccaccgatggccggttcctccaccagtggcggcggcggcgacgacgatgacgacgacggtgaggtcaagctctggagctactga

SEQ ID NO: 45 - DNA, 1993 bp, Zea mays
  CDS (136)...(804): ZmCBF4 genomic
ccgtagcacagcatttgttttcttacaatccttcctcgcaagtcgcatgccacacttacaaatacgcgccttccaaaccgccatgattcactccgattgcgcgccagatcaagctgcgagtcggcggcctcgatgatgccgccgccgccgtcctcgtcgtcttcgccctcgcaggacgccggcagccccaagcgagccgcggggcgcaacaagttccgggagacgcggcacccggtgttccgcggcgtgcgccggcgaggccgcgcggggggccggtggcggtgggtgtgcgaggtccgcgttccaggccgccgcggctgcaggctctggctcggcaccttcgcggccgcggaggccgccgcgcgcgcgcacgacgccgccatgctcgcgctccgcggcggcgccgcgcgcgcgcggtgcctcaacttcccggactcggcctggctgctggacgtgccggtgctgccgctgccccacggcgcagcgccctgggccgacgtccgccgcgccgtcgcgatagccgtcgaggggttcttccgggcgcggccagccgccgaggacgccatgtccgccacctcggagccgtcgtcagcggccacggaggcggaggcggaggcgtcctcctcctccgggaccgacggcggcgcgccggaggcctcgccgttcgagctggacatgctgagcgatatgggcgccggcttgtactacgcgtgcttagcgcaggggctgctcgtggagcctccaccgttagacgcgccgtgccccgacgacagcgactgtggcctcgcgctctggtcctactgaaacccatgtcgaccaacgcttgtagattatctattctttccttttgggaaatgcggatattataaattccacgaactagtacaatatttctctgttcacttcggaaatactagaaagttaccgaaatactgtacgatgcgcatgcgactggcagcaagccttttttccttttcttttctggaatgatgcaaatagaacgtgtagatgtttttagtccaggaatgttaagcgctaataattaatttcagtcacacttttcaacataaccaaactaaatggtacagtgctgagggctaacatttatttatttatttattaaactgatgcggctcgctcgccagacgtagcttccacgtcgacctaaaatcgtgtgtctgctgcagatatacatcttggccgcacacgtgtcttctaccgctatacattgcccttgtcttgcctagctgctgctttaccgtgggtccggaccgtgccgctcggaatttcccgcgaaaaaggaaaacaaaagcgtcagctggctcacccaagttaaaaagaaaaaaaaaacaatcacttcgatcttcaagatgctgctctgcttttggcaagctcgtagtgtaaatgtgtaattacctgattggcatagctaatgcctttgtgcaaaaacatattgcacaacctaagaacgtttactatatacctgtcttagccttttgctactccccacacggccacacctccaaccaggtgttcgtacactcccaatgctaccttaaagatatgttttatgtttgcgtcaatcatacctctacaaatgagtttgggtataatattatcctaaactatgggagatccattctccgagtttagggggtacaatagtttcctaaaaaaaataagagttcttgctaacctttctcatcttggtcttctgcagatgtcaacttcttataggctcgtccacctcggtgacgaccatgaaccggtgtagagcaagtggcgcgcaacgaataagcttgaaacgcgacccaaaccaaattcacgatgatttataggctcgtccgttatccacaccgacgtaagtatcaatctcaacttcgtattcgaatcacaacgcacagtagatgccctcatgtcgaggtccctgatgatcgtgctagcatgtttcagaagtatgaaactgaaacctttagccccccccccccccccc

SEQ ID NO: 46 - DNA, 4786 bp, Zea mays
  CDS (1700)...(2395): ZmCBF6 genomic
cgacgggtgcgggtatgagtgaagattttaacccgcgggcagccaacccgaagtttcgcgggtgcgtgtctctatttcaacccccgggtgacccacaaccgacccaaaatttggtgtgttcgttattttgtgcaataatgattaagatacaatattaattatttgtcaacaagtgacttggtcaatgattcaagaagtgttttgttgctgctcaagatgagaagacttaaaatacatagatatgactcaattattcgtggtatttgattgcttatagctgaattattgcttaaaaatgtacgatgagtaaaactcgatggtgtcccaaaacccacccgaaacctgatgggtttgggtgcgggtttagaattgcacccgcgggcgggtttagattaaactggtttttaggagttattttttgggagctcttggatatatgcttatggataagcatggaaaaacatatgatatgacgaccgtgagacagtaagtcctttgttgcagcatcaaccatcattcctgaagtactcacaagttttgcagccatcattcaacgagagaccacaattcatgctagtccggtccataattgactctaaaataatttaacagaacatatctgagatctatatcattccaattagattttattttatgaattgtatagttttacttgtttttacgtgatgtaattttattttttatgaattacgaaattttatttacttaattaagtactcggatgaagttttatgtcaactaaacgacgtgttccatgcaattaaaagttmgtraaagaaaaaaatgatgtggcgttcaggtaactgtagcttgagggtaaaggctgtgtactagagcacgatattagattaggactatgaagcttgataacagtgattgataaatgatatctaatataacggtcgataaaggctaaataaaaggttaacaataatcaatagcaacattgatagtatagtcgaagttgacgtgacggtcgctaaagatatcagcagaagcaatagaatcagagtaggagtggattgatttctgcagcagcaagcacggcggcgacgctctcgtagcacggcaggcgcgcaaattgcgggcgtacactgccaagccagcggtcgccatttatccatcaggccgtcgaacgaacgaagagcgccccgagaccaagtccaatataactgccacctgaccgtggagtgggatcctctgcgtggctccatccgatcacttgcactggtccagcgcgtacggcgtacagagaatcccacgctagctatccgtctcgcacgagtgcgcatcagactaccagagtttcgctctctcaaaggcgtcacgccttttcgttttcgacacacgtcgccgtcagtgaaagaaccgtaccacccaacagcagcgagggaggccagccggctgcgcgttccgcgagctccagctggttccgatccttcgagcgcgaagagacctctcgatctccagagtccagagtccatcagtcgcacaacctggctggcacctcccgagccgcaccgcgtaggaatagtgcacgcggccacgtccctctccacgcgctaacctatatagcccgccacctttccctccaaattcaaatcaaatgctccgactcgttcctccccctgctcgccacgcccacggccactaccactcacacacacaggcagcagccatggacgccgccggctccttcagcgactactcctctggaaccccgtcccctgtcgccggcggcggcggcggcgacgacttcggctcctcctcctacatgacagtgtcatcggcgccgcccaagcgccgagccgggcggaccaagttcaaggagacgcggcaccccgtgtacaagggcgtgcggcggaggaaccccgggaggtgggtctgcgaggtgcgggagccgcacggcaagcagcggatatggctcgggaccttcgagaccgccgagatggcggcgcgcgcgcacgacgtcgccgcgctcgcgctgcgcggccgcgccgcctgcctcaacttcgccgactcgccgcggctcctcagggtgcccccgacgggctccgggcacgacgagatacgccgcgcggccgccgtggcggcggaccagttccgcccggcgcccgatcagggcaatgtggccgccgaggaggaggcggccgatacaccaccaccggatgccttgcccagcgtgacgatgcagagcgtcgacgacgacccgtactgcattatcgacgacaggctcgacttcgggatgcaggggtacctcgacatggcgcaagggatgctcattgatccgccaccgatggccggttcctccaccagtggcggcggcggcgacgacgatgacgacgacggtgaggtcaagctctggagctactgatggccgatcgcacgtgtgtctgaaggaagacaggttgcattgggcaaataacttccctgtacagccttgggaagaaccggtaccggtgaaatgtactggccgtggccctttcccttcggttcgtctatgctatgtaatgttatgtatcctgctcttctgatgattaagggtattcaggagaagcagaagactgggtttactcggtttgatcgtttaatttaatttggagctagagatgtacctgcatgcatatataatgcatgcacggtaactgtgatataatattcagagtgccatcaacacctgtaacgagccataaaaagcatggtgtatgcatttaggaacgaccgtgggcatgatggtatactattgatgttgcattgagaatttggctcttgtcgcaggaatcagaaactagaatccttctaaaaaaaactggtacccacacgcacctgatcggctgatcatgaactccaatactctaagttcatgtgcttcaaatccttgctgtactgtaatgctacacggtaaatcataacgtggtcgctgtattttgatatgtatacttggagttattttttatatcacctaaagctatatctcgtgttttcttaagaaataaaataaggggtatgcatgaggtaactgttcgcttaaataatattctctctatcccaaaatataatttgttctaaactattcatatatatatttattaagtaacatatgaatatagttcacgtgtatgtctatattcattatcatttgaatgaacatggatggaaaaaaaagtaagctaaaaaaactatatttagaacggatggaatactgtatattgggtagacaatatgacatctcaaatataagaggatgagaggatagtttaagataccactaaggctctgttcggtttgtagggattggagccccggattgattcatagccggattacttctctaatttatatagattttgatgagctggaacgaatcctggttgattctcgtagaagcgaacgggccctaaatataggccaggatgaattgtagacgttttgactcttggacacaaagtttttgctatatgtctagacatatatcatgtatgtaagtgaataacaaaatgcattttcttggcaaacttcatatatatttttttctgcggtctatcaaatgattggtcattcaaaaatttctgtcttttcaatcctctatttttttacaactgtattaatacagaatttttttattttttctgttcatatgtctttgcattattctaaagaggctccaccggtctttttttaacgaatagaacattctgtctcgcattagcgcgagctcgtggtttgcgttgcagcttatgaaggctacggactacctgataacggaaaaggctttgacgatgtcaatgaaacggcgtagtgtagtgtagtgggctcagctggcaagccgcgggaaggaatactcggtccatagcatatagcgaggtactgaggtctcagcgtcactttcagagctcggagtggccgacgggtgatggtcactctcgcgcgctcactgggtcgggccgcaagtgccattccaaagccaacaaaagtagtgcccctgccaaccgccagctcacggagtcgcagcgtgtttctctcttttttacccagtcctcccgcgggttcgcgccgccgctgcatagcaaactagaagagaagttcctgtctgcgacttcaggagtcggaaacgctggcaagaaccacacgaacagcgggcagcaagtttttttttttttttatgttttcatttgattgtattggctcttgtcatgtaatgtactccctcactccgtctcaaaattaactagtgcgtcatctgaattctgaaccgaccgtatgagacggatcagatgatatgaacgctcaatccaaatgactatctggttaacggacatcatggtcctattaattcatgtttcataataatatataataatatagagatagtgttcaaataaagtcccaaaaaaagtgtctcgtcgagctagctatataggtcacatgcctatagcaatatattgtaccatccctcataccacaaacatatacaaccttattattttacggtttcatgtatcgtgttaggatatacatcaacaaattctcgatgtgtgtcaagctctataagtggtggcgatggaaatctttgtgtttcatggatgacgatcaaatgcaagacttgagtgtgcctagtgttttccatagccatgttatttggtttcgtcgagttagtttgatttttcaaagaactattcattaagtcaatatttgacatgtaatggatgtacgcatctaatacgaaaagccggaactcttgtttccataataaaaaaatgcatcg

(19) United States
(12) Patent Application Publication
(10) Pub. No.: US 2009/0188003 A1
(43) Pub. Date: Jul. 23, 2009

(54) Transcriptional Activators Involved in Abiotic Stress Tolerance

(75) Inventors: Shoba Sivasankar, Urbandale, IA (US); David A. Selinger, Johnston, IA (US); Norbert Brugiere, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 12/358,698

(22) Filed: Jan. 23, 2009

Related U.S. Application Data
(60) Provisional application No. 61/022,916, filed on Jan. 23, 2008.

(51) Int. Cl. C12N 15/11; A01H 5/00; C07H 21/04; C12N 15/63

(52) U.S. Cl. 800/278; 800/298; 800/320.1; 800/320; 800/320.3; 800/320.2; 800/314; 800/312; 800/322; 800/306; 800/317.3; 536/23.1; 435/320.1

(57) ABSTRACT

The present invention provides compositions and methods for regulating expression of nucleotide sequences in a plant. Compositions comprise novel polypeptides involved in modulating gene expression in response to abiotic stress such as cold or drought, and the polynucleotides encoding the polypeptides. Methods for expressing the polynucleotides in a plant and improving cold and/or drought tolerance of plants are also provided.

This application claims priority to, and hereby incorporates by reference in its entirety, U.S. Provisional Patent Application Ser. No. 61/022,916.

### FIELD OF THE INVENTION

The present invention relates to the field of plant molecular biology, more particularly to regulation of gene expression in plants.

### BACKGROUND OF THE INVENTION

Stresses to plants may be caused by both biotic and abiotic agents. For example, biotic causes of stress include infection with a pathogen, insect feeding, parasitism by another plant such as mistletoe, and grazing by ruminant animals. Abiotic stresses include, for example, excessive or insufficient available water, temperature extremes, synthetic chemicals such as herbicides, and excessive wind. Yet plants survive and often flourish, even under unfavorable conditions, using a variety of internal and external mechanisms for avoiding or tolerating stress. Plants' physiological responses to stress reflect changes in gene expression.

Insufficient water for growth and development of crop plants is a major obstacle to consistent or increased food production worldwide. Population growth, climate change, irrigation-induced soil salinity, and loss of productive agricultural land to development are among the factors contributing to a need for crop plants which can tolerate drought. Drought stress often results in reduced yield. In maize, this yield loss results in large part from plant failure to set and fill seed in the apical portion of the ear, a phenomenon known as tip kernel abortion.

Low temperatures can also reduce crop production. A sudden frost in spring or fall may cause premature tissue death.

Physiologically, the effects of drought and low-temperature stress may be similar, as both result in cellular dehydration. For example, ice formation in the intercellular spaces draws water across the plasma membrane, creating a water deficit within the cell. Thus, improvement of a plant's drought tolerance may improve its cold tolerance as well.

Plants adapt to environmental stresses such as cold, drought, and salinity through modulation of gene expression. Promoter regions of stress-inducible genes may comprise cis-acting elements, which are DNA fragments recognized by trans-acting factors. Transacting factors include, for example, proteins stimulated by abscisic acid (ABA) which bind to an ABA-responsive element (ABRE); see, for example, Yamaguchi-Shinozaki, et al., (2005) Trends in Plant Science 10(2):88-94. Transacting factors also include nuclear proteins capable of binding to regulatory DNA and interacting with other molecules, notably DNA Polymerase III, to initiate transcription of DNA operably linked to said regulatory DNA. Transcription factors may exist as families of related proteins that share a DNA-binding domain. The transcription factor genes may themselves be induced by stress. Furthermore, the downstream targets of cis-regulated genes may be transcription factors, creating a complex network of gene response cascades.

CBF genes (for C-repeat/DRE binding factor) encode proteins which may interact with a specific cis-acting element of certain plant promoters. (U.S. Pat. Nos. 5,296,462 and 5,356,816; Yamaguchi-Shinozaki, et al., (1994) The Plant Cell 6:251-264; Baker, et al., (1994) Plant Mol. Biol. 24:701-713; Jiang, et al., (1996) Plant Mol. Biol. 30:679-684) The cis-acting element is known as the C-repeat/DRE and typically comprises a 5-base-pair core sequence, CCGAC, present in one or more copies.

CBF proteins may comprise a CBF-specific domain and an AP2 domain and have been identified in various species, including Arabidopsis (Stockinger, et al., (1997) Proc. Natl. Acad. Sci. 94:1035-1040; Liu, et al., (1998) Plant Cell 10:1391-1406); Brassica napus, Lycopersicon esculentum, Secale cereale, and Triticum aestivum (Jaglo, et al., (2001) Plant Phys. 127:910-917) and Brassica juncea, Brassica oleracea, Brassica rapa, Raphanus sativus, Glycine max, and Zea mays (U.S. Pat. Nos. 6,417,428; 7,253,000 and 7,317,141).

DRE/CRT (Dehydration Response Element/C-Repeat) cis elements function in ABA-independent response to stress and have been identified in numerous plant species, including Arabidopsis, barley, Brassica, citrus, cotton, eucalyptus, grape, maize, melon, pepper, rice, soy, tobacco, tomato and wheat. The DRE/CRT elements comprise a core binding site, A/GCCGAC, recognized by the trans-activating factors known as DREB1 (DRE-Binding) and CBF (C-Repeat Binding Factor). Secondary structure in proximity to the cis element, and/or multiple cis factors appear to be additional components necessary for stress-inducible expression. (For reviews, see, Agarwal, et al., (2006) Plant Cell Rep 25:1263-1274; Yamaguchi-Shinozaki and Shinozaki, (2005) Trends in Plant Science 10(2):88-94). The promoter regions of the CBF/DREB genes may comprise cis-acting elements such as ICEr1 and ICEr2 (Zarka, et al., (2003) Plant Physiol. 133:910-918; Massari and Murre, (2000) Mol. Cell. Bio. 20:429-440).

Modification of complex agronomic traits requires the concurrent action of multiple genes belonging to multiple pathways. Use of single genes to modify complex agronomic traits may result in the realization of only part of the plant's potential to respond. In contrast, the CBF transcription factor presents an opportunity for overexpression of a single transcription factor to cause the simultaneous activation and overexpression of multiple downstream genes, to provide maximum possible modulation of the trait. The use of selected maize CBF genes based on expression analysis and association studies would enable informed targeting of transgenes or endogenous genes for transgenic modification, or marker-assisted breeding for abiotic stress tolerance.

Overexpression of CBF in plants has been shown to improve tolerance to drought, cold, and/or salt stress (Jaglo-Ottosen, et al., (1998) Science 280:104-106; Kasuga, et al., (1999) Nature Biotechnology 17:287-291; Hsieh, et al., (2002) Plant Phys. 129:1086-1094; Hsieh, et al., (2002) Plant Phys. 130:618-626; Dubouzet, et al., (2003) Plant J. 33:751-763). While CBF transcription factors may be useful in transgenic approaches to regulate plant response to stress, constitutive expression of CBF results in negative pleiotropic effects. Controlled expression of CBF in selected tissues and/or under stress conditions is of interest.

### SUMMARY OF THE INVENTION

Compositions and methods for regulating gene expression in a plant are provided. Compositions comprise isolated polypeptides involved in modulating gene expression in response to cold, salt, and/or drought, including SEQ ID NO: 13, 14, 15, 16, 17, 18, 19, 20, 21, 22, 23, 24, 25, 26, 27, 28 and 29. Further compositions of the invention comprise each polynucleotide encoding a polypeptide of the sequence set forth in SEQ ID NO: 13, 14, 15, 16, 17, 18, 19, 20, 21, 22, 23, 24, 25, 26, 27, 28 or 29, operable fragments of each, and sequences 85% identical to the full length coding sequence of each. The compositions of the invention further comprise polynucleotides set forth in SEQ ID NO: 30, 37, 38, 43 and 44, and full-length polynucleotides complementary thereto, as well as variants and fragments thereof. The sequences are referred to as CBF or CBF-like genes.

In one embodiment of the invention, a DNA construct comprises an isolated polynucleotide of the invention operably linked to a promoter sequence, wherein the promoter is capable of driving expression of the nucleotide sequence in a plant cell. The promoter sequence may be heterologous to the linked nucleotide sequence. In some embodiments, said promoter sequence is inducible by an exogenous agent or environmental condition. In some embodiments, said promoter initiates transcription preferentially in certain tissues or organs.

Also provided are expression cassettes comprising said DNA construct; vectors containing said expression cassette; transformed plant cells, transformed plants, and transformed seeds comprising the novel sequences of the invention.

Further embodiments comprise methods for expressing a polynucleotide or polypeptide of the invention in a plant. The methods comprise stably incorporating into the genome of a plant cell an expression cassette comprising a promoter sequence operably linked to a polynucleotide of the invention, wherein the promoter is capable of initiating transcription of said polynucleotide in a plant cell. Certain embodiments of the present invention comprise methods for modulating the development of a transformed plant under conditions of stress.

### BRIEF DESCRIPTION OF THE DRAWINGS

FIG. 1 provides an alignment of numerous CBF polypeptides from maize: ZmCBF7 (SEQ ID NO: 17), ZmCBF5 (SEQ ID NO: 15), ZmCBF8 (SEQ ID NO: 18), ZmCBF2 (SEQ ID NO: 2, also noted herein as 1084 SEQ 2), ZmCBF10 (SEQ ID NO: 20), ZmCBF4 (SEQ ID NO: 14), ZmCBF9 (SEQ ID NO: 19), ZmCBF11 (SEQ ID NO: 21), ZmCBF6 (SEQ ID NO: 16), ZmCBF1 (SEQ ID NO: 4, also noted herein as 1084 SEQ 4), ZmCBF3 (SEQ ID NO: 13), ZmCBF16 (SEQ ID NO: 26), ZmCBF15 (SEQ ID NO: 25), ZmCBF17 (SEQ ID NO: 27), ZmCBF19 (SEQ ID NO: 29), ZmCBF12 (SEQ ID NO: 22), ZmCBF13 (SEQ ID NO: 23), ZmCBF14 (SEQ ID NO: 24), ZmCBF18 (SEQ ID NO: 28).

FIG. 2 provides a dendogram of the sequences aligned in FIG. 1. Both FIGS. 1 and 2 were created using PileUp software from Accelrys, Inc. at default settings (blosum 62 scoring matrix; gap creation penalty of 8; gap extension penalty of 2; maximum input sequence range, 5000; maximum number of gap characters added, 2000). Note that ZmCBF2 (SEQ ID NO: 2) is shown as 1084 SEQ 2; ZmCBF1 (SEQ ID NO: 4) is shown as 1084 SEQ 4.

FIG. 3 is a portion of the alignment of FIG. 1 wherein the AP2 domain is underlined and the CBF-specific domain is in bold font, for ZmCBF1, ZmCBF2, and ZmCBF3.

FIG. 4 is a table of expression profiling results for ZmCBF3 through ZmCBF9 and ZmCBF11.

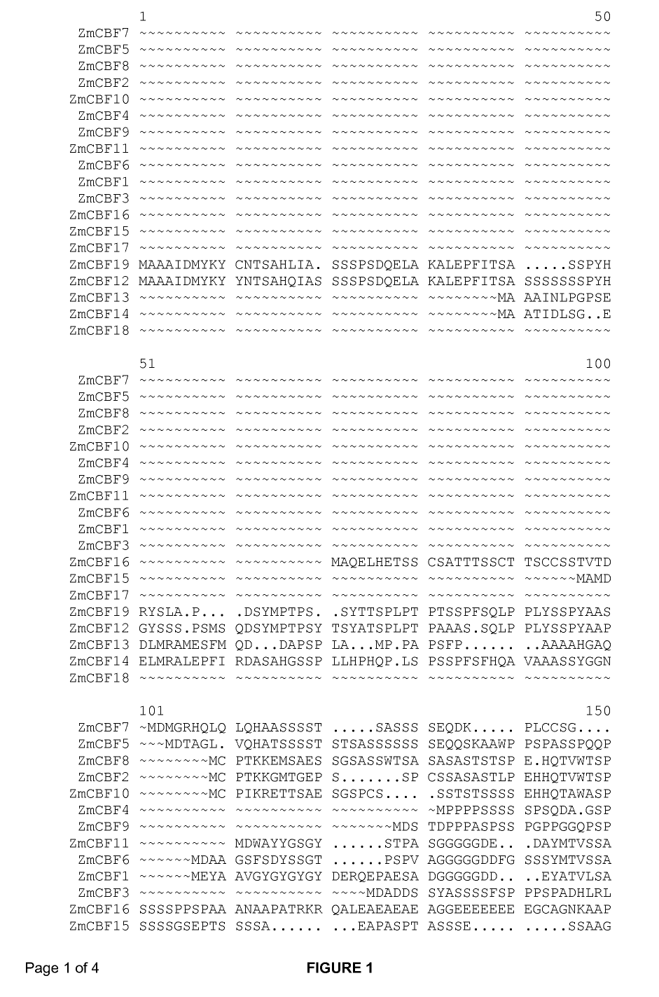

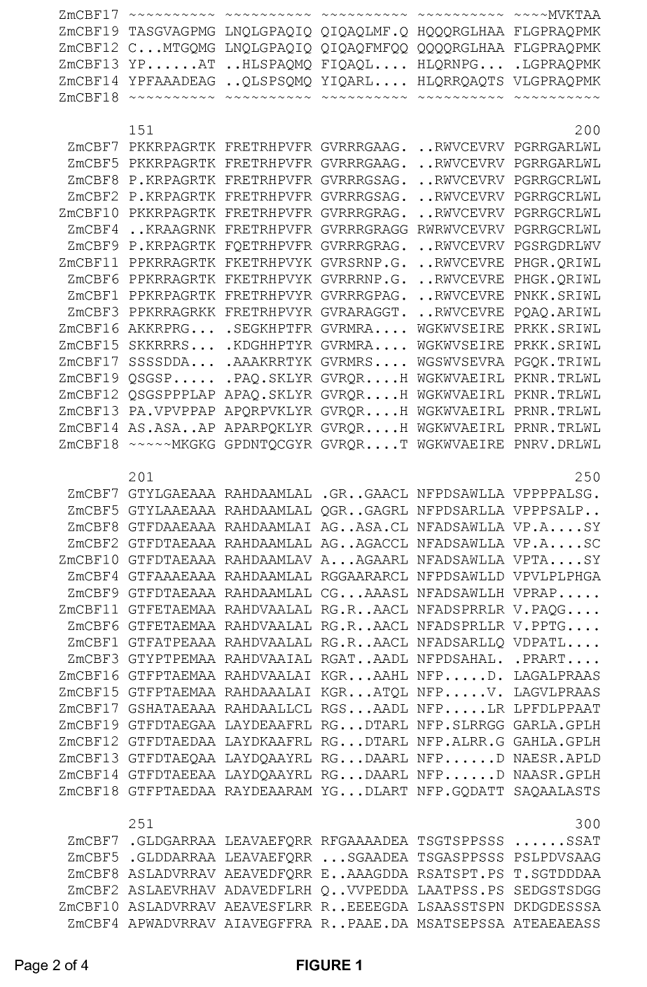

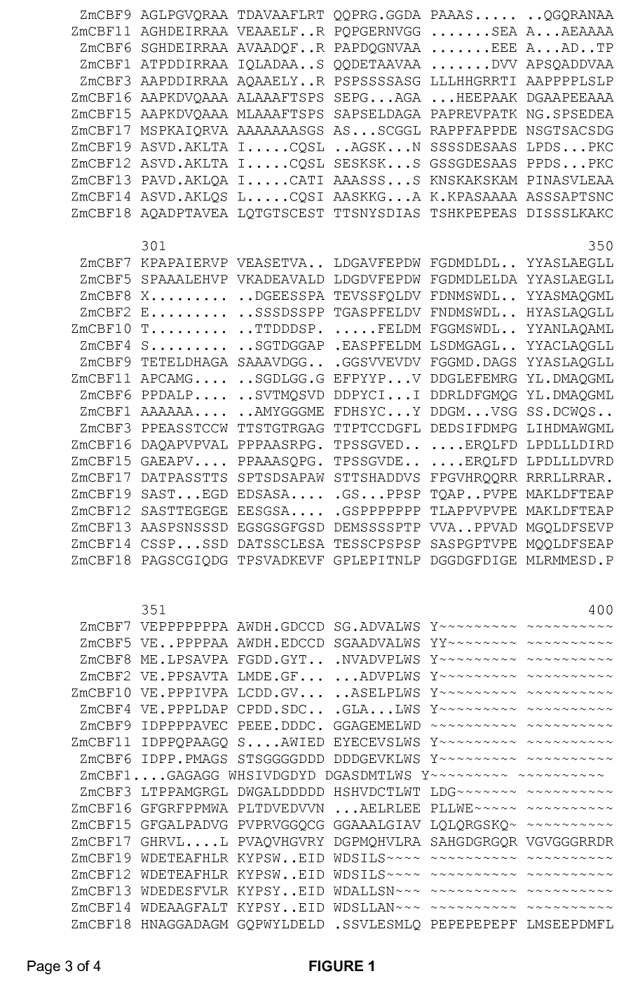

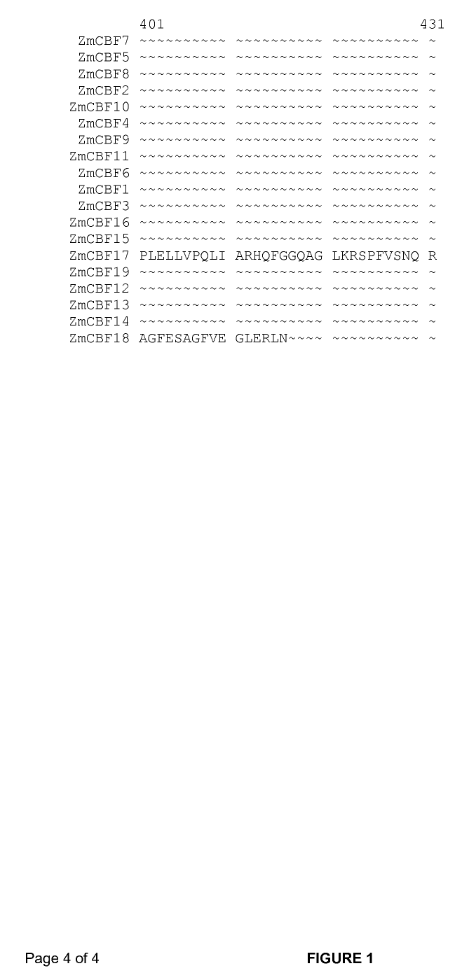

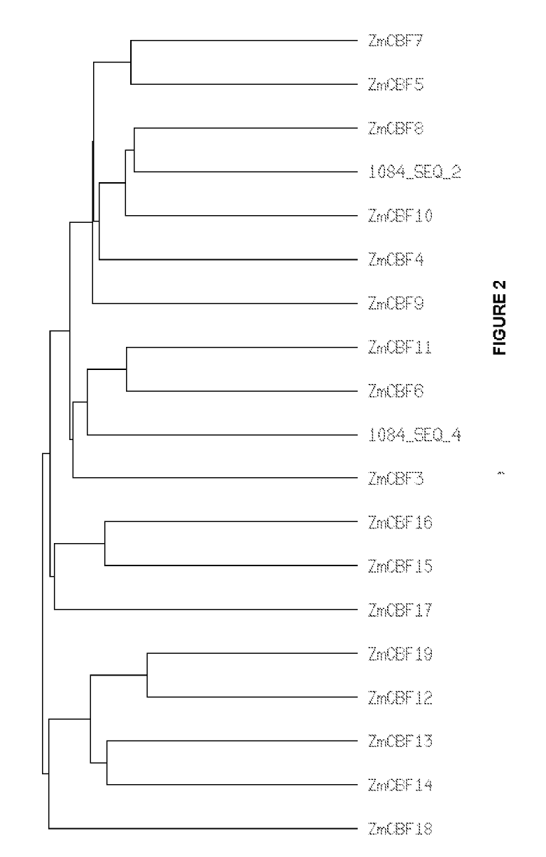

BRIEF DESCRIPTION OF THE SEQUENCES

| | SEQ ID NO: in | SEQ ID NO: in | SEQ ID NO: in | SEQ ID NO: in |
|---|---|---|---|---|
| | this application | 61/022,916 | U.S. Pat. No. 7,253,000 | U.S. Pat. No. 7,317,141 |
| ZmCBF2 | 1 & 2 | 1 & 2 | 1 & 2 | 1 & 2 |
| ZmCBF1 | 3 & 4 | 3 & 4 | 3 & 4 | 3 & 4 |
| Zm Rab17 promoter | 5 | 5 | 5 | 5 |
| Arabidopsis rd29a | 6 | 6 | 6 | 6 |
| promoter | | | | |
| Zm RIP2 promoter | 7 & 8 | 7 & 8 | 7 & 8 | 7 & 8 |
| Zm mLIP15 promoter | 9 | 9 | 9 | 9 |
| Rye CBF31 | 10 | 10 | 10 | 10 |
| Arabidopsis CBF3 | 11 & 12 | 11 & 12 | 11 & 12 | 11 & 12 |
| ZmCBF3 | 13 & 30 | 13 & 30 | N/A | N/A |
| ZmCBF4 | 14, 43 & 45 | 14 | N/A | N/A |
| ZmCBF5 | 15 & 31 | 15 & 31 | N/A | N/A |
| ZmCBF6 | 16, 44 & 46 | 16 | N/A | N/A |
| ZmCBF7 | 17 | 17 | N/A | N/A |
| ZmCBF8 | 18 | 18 | N/A | N/A |
| ZmCBF9 | 19 | 19 | N/A | N/A |
| ZmCBF10 | 20 | 20 | N/A | N/A |
| ZmCBF11 | 21 | 21 | N/A | N/A |
| ZmCBF12 | 22, 32 & 33 | 22, 32 & 33 | N/A | N/A |
| ZmCBF13 | 23, 34 & 35 | 23, 34 & 35 | N/A | N/A |
| ZmCBF14 | 24 & 36 | 24 & 36 | N/A | N/A |
| ZmCBF15 | 25 & 37 | 25 & 37 | N/A | N/A |
| ZmCBF16 | 26 & 38 | 26 & 38 | N/A | N/A |
| ZmCBF17 | 27 & 39 | 27 & 39 | N/A | N/A |
| ZmCBF18 | 28 & 40 | 28 & 40 | N/A | N/A |
| ZmCBF19 | 29 & 41 | 29 & 41 | N/A | N/A |
| RyeCBF31 promoter | 42 | 42 | N/A | N/A |

### DETAILED DESCRIPTION OF THE INVENTION

The invention provides isolated polypeptides active as transcription initiation factors involved in stress-induced gene expression, particularly drought or cold stress.

By “recombinant expression cassette” or “expression cassette” is meant a nucleic acid construct, generated recombinantly or synthetically, comprising a series of specified nucleic acid elements which permit transcription of a particular nucleic acid in a host cell. The recombinant expression cassette can be incorporated into a plasmid, chromosome, mitochondrial DNA, plastid DNA, virus or nucleic acid fragment. Typically, the expression cassette portion of an expression vector includes, among other sequences, a promoter and a nucleic acid to be transcribed. A polynucleotide sequence encoding ZmCBF3 is provided at SEQ ID NO: 30. A polynucleotide sequence encoding ZmCBF4 is provided at SEQ ID NO: 43. A polynucleotide sequence encoding ZmCBF6 is provided at SEQ ID NO: 44. A polynucleotide sequence encoding ZmCBF15 is provided at SEQ ID NO: 37. A polynucleotide sequence encoding ZmCBF17 is provided at SEQ ID NO: 39. Other polynucleotide coding sequences can be derived by a person of skill in the art from the amino acid sequences provided.

By “heterologous nucleotide sequence” is intended a sequence that is not naturally occurring with another sequence. For example, a nucleotide sequence encoding a transcription factor may be heterologous to the promoter sequence to which it is operably linked. Further, the coding sequence and/or the promoter sequence may be native or foreign to the plant host.

By “operable fragment” is meant a truncated or altered form of a particular polynucleotide or polypeptide which is sufficient to perform or provide the relevant function. For example, where the goal is to interfere with gene function, a truncated form of a polynucleotide may be sufficient for purposes of co-suppression or anti-sense regulation. Where the goal is to initiate transcription, a promoter or transcription factor which is less than the full length known, or which comprises minimal internal deletions or alterations, may still function appropriately. Promoter sequences provided, or one or more fragments thereof, may be used either alone or in combination with other sequences to create synthetic promoters. In such embodiments, the fragments (also called “cis-acting elements” or “subsequences”) confer desired properties on the synthetic promoter.

By “promoter” is intended a region of DNA upstream from the start of transcription and involved in recognition and binding of RNA polymerase and other proteins to initiate transcription. A promoter usually comprises a TATA box capable of directing RNA polymerase II to initiate RNA synthesis at the appropriate transcription initiation site for a particular coding sequence. A promoter can additionally comprise other recognition sequences generally positioned upstream or 5′ to the TATA box, referred to as upstream promoter elements, which influence the transcription initiation rate. Thus a promoter region may be further defined by comprising upstream regulatory elements such as those responsible for tissue and temporal expression of the coding sequence, enhancers, and the like. In the same manner, the promoter elements which enable expression in the desired tissue can be identified, isolated, and used with other core promoters.

A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells, such as Agrobacterium or Rhizobium. Examples of promoters under developmental control include tissue-preferred promoters, which preferentially initiate transcription in certain tissues, such as leaves, roots, or seeds, and those promoters driving expression when a certain physiological stage of development is reached, such as senescence. Promoters which initiate transcription only in certain tissue are referred to as “tissue-specific.” A “cell-type-preferred” promoter primarily drives expression in certain cell types in one or more organs, for example, vascular tissue in roots or leaves. An “inducible” or “repressible” promoter is a promoter which is under environmental control. Examples of environmental conditions that may effect transcription by inducible promoters include anaerobic conditions or the presence of light. Certain promoters are induced by unfavorable environmental conditions, for example, rab17 (exemplified by SEQ ID NO: 5; see also, Busk, et al., (1997) Plant J 11:1285-1295), rd29A (exemplified by SEQ ID NO: 6; see also, GenBank D13044 and Plant Cell 6:251-264, (1994)), rip2 (exemplified by SEQ ID NOS: 7 and 8; see also, GenBank L26305 and Plant Phys. 107(2):661-662 (1995)), mlip15 (exemplified by SEQ ID NO: 9; see also, GenBank D63956; Mol. Gen. Gen. 248(5):507-517 (1995); and ryeCBF31 (U.S. Patent Application Ser. No. 60/981,861 filed Oct. 23, 2007). Tissue-specific, tissue-preferred, cell-type-preferred and inducible promoters are members of the class of “non-constitutive” promoters. A “constitutive” promoter is a promoter which is active in all or nearly all tissues, at all or nearly all developmental stages, under most environmental conditions.

It is recognized that to increase transcription levels, enhancers can be utilized in combination with promoter regions to increase expression. Enhancers are known in the art and include the SV40 enhancer region, the 35S enhancer element, and the like.

A “subject plant” or “subject plant cell” is one in which genetic alteration, such as transformation, has been affected as to a gene of interest, or is a plant or plant cell which is descended from a plant or plant cell so altered and which comprises the alteration. A “control” or “control plant” or “control plant cell” provides a reference point for measuring changes in the subject plant or plant cell.

A control plant or control plant cell may comprise, for example: (a) a wild-type plant or plant cell, i.e., of the same genotype as the starting material for the genetic alteration which resulted in the subject plant or subject plant cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or subject plant cell; (d) a plant or plant cell genetically identical to the subject plant or subject plant cell but which is not exposed to conditions or stimuli that would induce expression of the gene of interest; or (e) the subject plant or subject plant cell itself, under conditions in which the gene of interest is not expressed.

The term “isolated” refers to material, such as a nucleic acid or a protein, which is: (1) substantially or essentially free from components which normally accompany or interact with it as found in its natural environment. The isolated material optionally comprises material not found with the material in its natural environment; or (2) if the material is in its natural environment, the material has been synthetically altered or synthetically produced by deliberate human intervention and/or placed at a different location within the cell. The synthetic alteration or creation of the material can be performed on the material within or apart from its natural state. For example, a naturally-occurring nucleic acid becomes an isolated nucleic acid if it is altered or produced by non-natural, synthetic methods, or if it is transcribed from DNA which has been altered or produced by non-natural, synthetic methods. The isolated nucleic acid may also be produced by the synthetic re-arrangement (“shuffling”) of a part or parts of one or more allelic forms of the gene of interest. Likewise, a naturally-occurring nucleic acid (e.g., a promoter) becomes isolated if it is introduced to a different locus of the genome.

A polynucleotide may be single- or double-stranded, depending on the context, and one of skill in the art would recognize which construction of the term is appropriate.

The Zea mays sequences of the invention can be used to isolate corresponding sequences from other organisms, particularly from other plants, more particularly from other monocotyledonous plants. Methods such as PCR, hybridization, and the like can be used to identify such sequences based on their similarity to a sequence set forth herein. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the sequences of the invention. For example, an entire sequence disclosed herein, or one or more portions thereof, may be used as a probe capable of specifically hybridizing to corresponding sequences. To achieve specific hybridization under a variety of conditions, such probes include sequences that are distinctive and are at least about 10 nucleotides in length. The well-known process of polymerase chain reaction (PCR) may be used to isolate or amplify additional sequences from a chosen organism or as a diagnostic assay to determine the presence of corresponding sequences in an organism. Hybridization techniques include hybridization screening of plated DNA libraries (either plaques or colonies; see, for example, Sambrook, et al., supra; see also, Innis, et al., eds., (1990) PCR Protocols, A Guide to Methods and Applications, Academic Press). Methods for preparation of probes for hybridization and for construction of cDNA and genomic libraries are generally known in the art and are disclosed in Sambrook, et al., (1989) Molecular Cloning: A Laboratory Manual (2d ed., Cold Spring Harbor Laboratory Press, Plainview, N.Y.) and Ausubel, et al., eds. (1995) Current Protocols in Molecular Biology, Chapter 2 (Greene Publishing and Wiley-Interscience, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are target-sequence-dependent and will differ depending on the structure of the polynucleotide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringency may also be adjusted with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)—0.61 (% form)—500/L; where M is the molarity of monovalent cations, % GC is the percentage of guanine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel, et al., eds. (1995) Current Protocols in Molecular Biology, Chapter 2 (Greene Publishing and Wiley-Interscience, New York). See also, Sambrook, et al., (1989) Molecular Cloning: A Laboratory Manual (2d ed., Cold Spring Harbor Laboratory Press, Plainview, N.Y.). Thus, isolated sequences that retain the function of the invention and hybridize under stringent conditions to the sequences disclosed herein, or to their complements, or to fragments of either, are encompassed by the present invention. Such a sequence will usually be at least about 85% identical to a disclosed sequence. That is, the identity of sequences may range, sharing at least about 85%, 86%, 87%, 88%, 89%, 90%, 91 %, 92%, 93%, 94%, 95%, 96%, 97%, 98%, 99% or more sequence identity.

Methods of alignment of sequences for comparison are well-known in the art. Optimal alignment of sequences for comparison may be conducted by the local homology algorithm of Smith and Waterman, (1981) Adv. Appl. Math. 2:482; by the homology alignment algorithm of Needleman and Wunsch, (1970) J. Mol. Biol. 48:443; by the search for similarity method of Pearson and Lipman, (1988) Proc. Natl. Acad. Sci. 85:2444; by computerized implementations of these algorithms, including, but not limited to: CLUSTAL in the PC/Gene program by Intelligenetics, Mountain View, Calif.; PileUp, GAP, BESTFIT, BLAST, FASTA and TFASTA in the GCG® Wisconsin Package™ from Accelrys, Inc., San Diego, Calif.

The CLUSTAL program is well described by Higgins and Sharp, (1988) Gene 73:237-244; Higgins and Sharp, (1989) CABIOS 5:151-153; Corpet, et al., (1988) Nucleic Acids Research 16:10881-90; Huang, et al., (1992) Computer Applications in the Biosciences 8:155-65, and Pearson, et al., (1994) Methods in Molecular Biology 24:307-331. A description of BLAST (Basic Local Alignment Search Tool) is provided by Altschul, et al., (1993) J. Mol. Biol. 215:403-410.

Identity to the sequence of the present invention would mean a polypeptide sequence having at least 85% sequence identity, wherein the percent sequence identity is based on the entire length of SEQ ID NO: 13, 14, 15, 16, 17, 18, 19, 20, 21, 22, 23, 24, 25, 26, 27, 28 or 29.

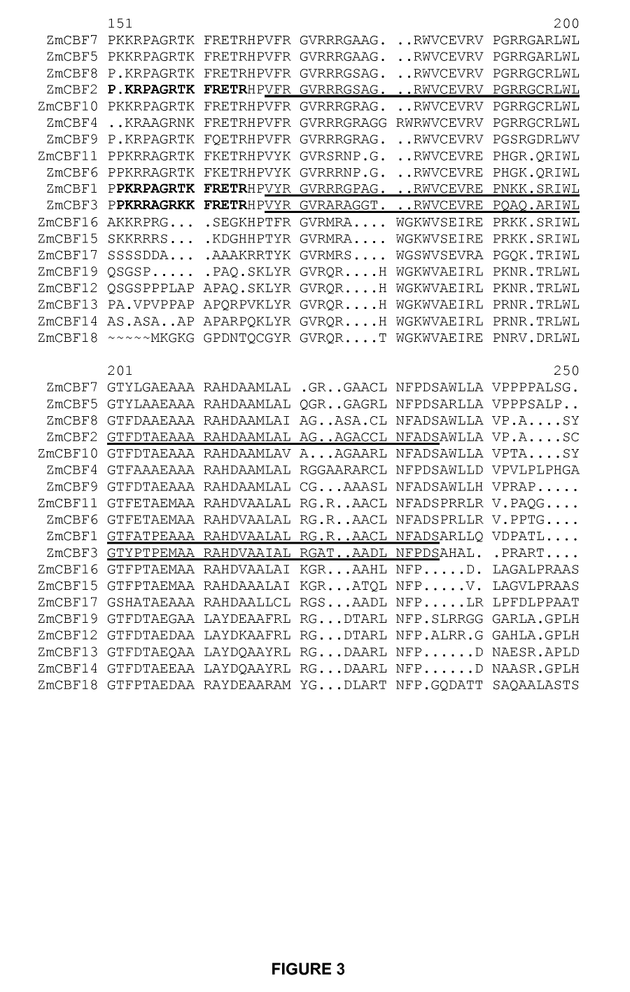

The AP2 domain is highly conserved among CBF genes, and some species share an additional conserved region bracketing the AP2 domains. (Jaglo, et al., (2001) Plant Phys. 127:910-917). For example, in FIG. 3, the AP2 domain of ZmCBF1, ZmCBF2 and ZmCBF3 is underlined. The CBF-specific domain of the same sequences is in bold font. Thus one of skill in the art would recognize that variants most likely to retain function are those in which at least one domain is undisturbed.

The invention encompasses isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5% or 1% (by dry weight) of contaminating protein. When the protein of the invention or biologically active portion thereof is recombinantly produced, optimally culture medium represents less than about 30%, 20%, 10%, 5% or 1% (by dry weight) of chemical precursors or non-protein-of-interest chemicals.

Fragments and variants of ZmCBF polynucleotides and proteins are also encompassed by the methods and compositions of the present invention. By “fragment” is intended a portion of the polynucleotide or a portion of the amino acid sequence. Fragments of a polynucleotide may encode protein fragments that retain the biological activity of the native protein and hence regulate transcription. For example, polypeptide fragments may comprise the CBF-specific domain or the AP2 domain. In some embodiments, the polypeptide fragment will comprise both the CBF-specific domain and the AP2 domain. Alternatively, fragments that are used for suppressing or silencing (i.e., decreasing the level of expression) of a CBF sequence need not encode a protein fragment, but will retain the ability to suppress expression of the target sequence. In addition, fragments that are useful as hybridization probes generally do not encode fragment proteins retaining biological activity. Thus, fragments of a nucleotide sequence may range from at least about 11 nucleotides, about 20 nucleotides, about 50 nucleotides, about 100 nucleotides and up to the full-length polynucleotide encoding a protein of the invention.

A fragment of a polynucleotide encoding a CBF-specific or AP2 domain or a CBF polypeptide will encode at least 14, 25, 30, 50, 60, 70, 100, 150, 200, 250 or 300 contiguous amino acids, or up to the total number of amino acids present in a full-length CBF-specific or AP2 domain, or CBF or CBF-like protein. Fragments of an AP2 or CBF-specific domain, or a CBF or CBF-like polynucleotide that are useful as hybridization probes, PCR primers, or as suppression constructs generally need not encode a biologically active portion of a CBF protein.

A biologically active portion of a polypeptide comprising an AP2 or CBF-specific domain, or a CBF or CBF-like protein, can be prepared by isolating a portion of a CBF-like polynucleotide, expressing the encoded portion of the CBF-like protein (e.g., by recombinant expression in vitro), and assessing the activity of the encoded portion of the CBF-like protein. A polynucleotide that is a fragment of a CBF-like nucleotide sequence, or a polynucleotide sequence comprising an AP2 or CBF-specific domain, comprises at least 42, 75, 100, 150, 200, 250, 300, 350, 400, 450, 500, 550, 600, 650, 700, 800, 900, 1000, 1100, 1200, 1300, 1400 or 1,500 contiguous nucleotides, or up to the number of nucleotides present in a full-length AP2 or CBF-specific domain or in a CBF-like polynucleotide.

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the CBF-like polypeptides or of an AP2 or a CBF-specific domain. Naturally occurring allelic variants such as these can be identified with the use of well-known molecular biology techniques, as, for example, with polymerase chain reaction (PCR) and hybridization techniques as outlined elsewhere herein. Variant polynucleotides also include synthetically derived polynucleotides, such as those generated, for example, by using site-directed mutagenesis but which still encode a polypeptide comprising an AP2 or a CBF-specific domain (or both), or a CBF-like polypeptide that is capable of regulating transcription or that is capable of reducing the level of expression (i.e., suppressing or silencing) of a CBF-like polynucleotide. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 13 is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they continue to possess the desired biological activity of the native protein, that is, regulate transcription as described herein. Such variants may result from, for example, genetic polymorphism or human manipulation. Biologically active variants of a CBF-like protein of the invention or of an AP2 or CBF-specific domain will have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the CBF-like protein or the consensus AP2 or CBF-like domain as determined by sequence alignment programs and parameters described elsewhere herein. A biologically active variant of a CBF-like protein of the invention or of an AP2 or CBF domain may differ from that protein by as few as 1-15 amino acid residues, as few as 1-10, such as 6-10, as few as 5, as few as 4, 3, 2 or even by one amino acid residue.

The polypeptides of the invention may be altered in various ways including amino acid substitutions, deletions, truncations, and insertions. Methods for such manipulations are generally known in the art. For example, amino acid sequence variants and fragments of the CBF-like proteins or AP2 or CBF-like domains can be prepared by mutations in the encoding DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired activity (i.e., the ability to regulate transcription). In specific embodiments, the mutations that will be made in the DNA encoding the variant do not place the sequence out of reading frame and do not create complementary regions that could produce secondary mRNA structure. See, EP Patent Publication Number 0075444.

The deletions, insertions, and substitutions of the protein sequences encompassed herein are not expected to produce radical changes in the characteristics of the protein. However, when it is difficult to predict the exact effect of the substitution, deletion, or insertion, one skilled in the art will appreciate that the effect will be evaluated by routine screening assays. For example, the activity of a CBF-like polypeptide can be evaluated by assaying for the ability of the polypeptide to regulate transcription. Various methods can be used to assay for this activity, including, directly monitoring the level of expression of a target gene at the nucleotide or polypeptide level. Methods for such an analysis are known and include, for example, Northern blots, S1 protection assays, Western blots, enzymatic or colorimetric assays. In specific embodiments, determining if a sequence has CBF-like activity can be assayed by monitoring for an increase or decrease in the level or activity of a target gene. Alternatively, methods to assay for a modulation of transcriptional activity can include monitoring for an alteration in the phenotype of the plant. For example, as discussed in further detail elsewhere herein, modulating the level of a CBF-like polypeptide can result in altered plant tolerance to abiotic stress. Methods to assay for these changes are discussed in further detail elsewhere herein.

Variant polynucleotides and proteins also encompass sequences and proteins derived from a mutagenic and recombinogenic procedure such as DNA shuffling. With such a procedure, one or more different CBF-like coding sequences can be manipulated to create a new CBF-like sequence or AP2 or CBF-specific domain possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the CBF-like gene of the invention and other known CBF-like genes to obtain a new gene coding for a protein with an improved property of interest, such as an increased Kₘ in the case of an enzyme. Strategies for such DNA shuffling are known in the art. See, for example, Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291; and U.S. Pat. Nos. 5,605,793 and 5,837,458.

The expression cassette may also include, at the 3′ terminus of the heterologous nucleotide sequence of interest, a transcriptional and translational termination region functional in plants. The termination region can be native with the promoter nucleotide sequence present in the expression cassette, can be native with the DNA sequence of interest, or can be derived from another source. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., 1989) Nucleic Acids Res. 17:7891-7903; Joshi, et al., (1987) Nucleic Acid Res. 15:9627-9639.

The expression cassettes can additionally contain 5′ leader sequences. Such leader sequences can act to enhance translation. Translation leaders are known in the art and include: picornavirus leaders, for example, EMCV leader (Encephalomyocarditis 5′ noncoding region), Elroy-Stein, et al., (1989) Proc. Nat. Acad. Sci. USA 86:6126-6130; potyvirus leaders, for example, TEV leader (Tobacco Etch Virus), Allison, et al., (1986); MDMV leader (Maize Dwarf Mosaic Virus), Virology 154:9-20; human immunoglobulin heavy-chain binding protein (BiP), Macejak, et al., (1991) Nature 353:90-94; untranslated leader from the coat protein mRNA of alfalfa mosaic virus (AMV RNA 4), Jobling, et al., (1987) Nature 325:622-625); tobacco mosaic virus leader (TMV), Gallie, et al., (1989) Molecular Biology of RNA, pages 237-256; and maize chlorotic mottle virus leader (MCMV) Lommel, et al., (1991) Virology 81:382-385. See also, Della-Cioppa, et al., (1987) Plant Physiology 84:965-968. The cassette can also contain sequences that enhance translation and/or mRNA stability such as introns.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, and the like.

In preparing the expression cassette, the various DNA fragments can be manipulated so as to provide for the DNA sequences in the proper orientation and, as appropriate, in the proper reading frame. Toward this end, adapters or linkers can be employed to join the DNA fragments, or other manipulations can be involved to provide for convenient restriction sites, removal of superfluous DNA, removal of restriction sites, or the like. For this purpose, in vitro mutagenesis, primer repair, restriction digests, annealing, and resubstitutions, such as transitions and transversions, can be involved.

As noted herein, the present invention provides vectors capable of expressing the claimed sequences under the control of an operably linked promoter. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook, et al., (supra).

The transformation vector, comprising a sequence of the present invention operably linked to a promoter in an expression cassette, can also contain at least one additional nucleotide sequence for a gene to be cotransformed into the organism. Alternatively, the additional sequence(s) can be provided on another transformation vector.

Vectors that are functional in plants can be binary plasmids derived from Agrobacterium. Such vectors are capable of transforming plant cells. These vectors contain left and right border sequences that are required for integration into the host (plant) chromosome. At a minimum, between these border sequences is the gene to be expressed under control of an operably-linked promoter. In preferred embodiments, a selectable marker and a reporter gene are also included. For ease of obtaining sufficient quantities of vector, a bacterial origin that allows replication in E. coli is preferred.

Reporter genes can be included in the transformation vectors. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) BioTechniques 19:650-655; and Chiu, et al., (1996) Current Biology 6:325-330.

Selectable marker genes for selection of transformed cells or tissues can be included in the transformation vectors. These can include genes that confer antibiotic resistance or resistance to herbicides. Examples of suitable selectable marker genes include, but are not limited to, genes encoding resistance to chloramphenicol, Herrera Estrella, et al., (1983) EMBO J. 2:987-992; methotrexate, Herrera Estrella, et al., (1983) Nature 303:209-213; Meijer, et al., (1991) Plant Mol. Biol. 16:807-820; hygromycin, Waldron, et al., (1985) Plant Mol. Biol. 5:103-108; Zhijian, et al., (1995) Plant Science 108:219-227; streptomycin, Jones, et al., (1987) Mol. Gen. Genet. 210:86-91; spectinomycin, Bretagne-Sagnard, et al., (1996) Transgenic Res. 5:131-137; bleomycin, Hille, et al., (1990) Plant Mol. Biol. 7:171-176; sulfonamide, Guerineau, et al., (1990) Plant Mol. Biol. 15:127-136; bromoxynil, Stalker, et al., (1988) Science 242:419-423; glyphosate, Shaw, et al., (1986) Science 233:478-481; phosphinothricin, DeBlock, et al., (1987) EMBO J. 6:2513-2518.

Other genes that could serve utility in the recovery of transgenic events but might not be required in the final product would include, but are not limited to, examples such as GUS (β-glucuronidase), Jefferson (1987) Plant Mol. Biol. Rep. 5:387); GFP (green fluorescence protein), Chalfie, et al., (1994) Science 263:802, and Gerdes (1996) FEBS Lett. 389:44-47; DSred (Dietrich, et al., (2002) Biotechniques 2(2):286-293); luciferase, Teeri, et al., (1989) EMBO J. 8:343; KN1 (Smith, et al., (1995) Dev. Genetics 16(4):344-348); Sugary1, Rahman, et al., (1998) Plant Physiol. 117:425-435; James, et al., (1995) Plant Cell 7:417-429 and GenBank Accession Number U18908; and systems utilizing the maize genes encoding enzymes for anthocyanin production, including CRC, P (Bruce, et al., (2000) Plant Cell 12(1):65-79, and R (Ludwig, et al., (1990) Science 247:449).

The transformation vector comprising an isolated polynucleotide encoding a polypeptide of the present invention, operably linked to a promoter sequence in an expression cassette, can be used to transform any plant. In this manner, genetically modified plants, plant cells, plant tissue, seed, and the like can be obtained. Transformation protocols can vary depending on the type of plant or plant cell targeted for transformation, e.g., monocot or dicot. Suitable methods of transforming plant cells include microinjection, Crossway, et al., (1986) Biotechniques 4:320-334; electroporation, Riggs, et al., (1986) Proc. Natl. Acad. Sci. USA 83:5602-5606; Agrobacterium-mediated transformation, see for example, Townsend, et al., U.S. Pat. No. 5,563,055; direct gene transfer, Paszkowski, et al., (1984) EMBO J. 3:2717-2722; and ballistic particle acceleration, see for example, Sanford, et al., U.S. Pat. No. 4,945,050; Tomes, et al., (1995) in Plant Cell, Tissue, and Organ Culture: Fundamental Methods, ed. Gamborg and Phillips, (Springer-Verlag, Berlin); and McCabe, et al., (1988) Biotechnology 6:923-926. Also see, Weissinger, et al., (1988) Annual Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); Klein, et al., (1988) Plant Physiol. 91:440-444 (maize); Fromm, et al., (1990) Biotechnology 8:833-839; Hooydaas-Van Slogteren, et al., (1984) Nature (London) 311:763-764; Bytebier et al. (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418; and Kaeppler, et al., (1992) Theor. Appl. Genet. 84:560-566 (whisker-mediated transformation); D. Halluin, et al., (1992) Plant Cell 4:1495-1505 (electroporation); Li, et al., (1993) Plant Cell Reports 12:250-255 and Christou, et al., (1995) Annals of Botany 75:407-413 (rice); Osjoda, et al., (1996) Nature Biotechnology 14:745-750 (maize via Agrobacterium tumefaciens); all of which are herein incorporated by reference.

The cells that have been transformed can be grown into plants in accordance with conventional ways. See, for example, McCormick, et al., (1986) Plant Cell Reports 5:81-84. These plants can then be pollinated with the same transformed strain or different strains. The resulting plants having expression of the desired characteristic can then be identified. Two or more generations can be grown to ensure that the desired phenotypic characteristic is stably maintained and inherited under conditions of interest.

In certain embodiments the nucleic acid sequences of the present invention can be used in combination (“stacked”) with other polynucleotide sequences of interest in order to create plants with a desired phenotype. The polynucleotides of the present invention may be stacked with any gene or combination of genes, and the combinations generated can include multiple copies of any one or more of the polynucleotides of interest. The desired combination may affect one or more traits; that is, certain combinations may be created for modulation of gene expression involved in plant response to stress. Other combinations may be designed to produce plants with a variety of desired traits, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106; and WO 98/20122); and high methionine proteins (Pedersen, et al., (1986) J. Biol. Chem. 261:6279; Kirihara, et al., (1988) Gene 71:359; and Musumura, et al., (1989) Plant Mol. Biol. 12: 123)); increased digestibility (e.g., modified storage proteins (U.S. patent application Ser. No. 10/053,410, filed Nov. 7, 2001); and thioredoxins (U.S. patent application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529 ); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility (e.g., see, U.S. Pat. No. 5.583,210), stalk strength, flowering time, or transformation technology traits such as cell cycle regulation or gene targeting (e.g., WO 99/61619; WO 00/17364; WO 99/25821), the disclosures of which are herein incorporated by reference.

These stacked combinations can be created by any method, including but not limited to cross breeding plants by any conventional or TopCross methodology, or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences of interest can be driven by the same promoter or by different promoters. In certain cases, it may be desirable to introduce a transformation cassette that will suppress the expression of a polynucleotide of interest. This may be accompanied by any combination of other suppression cassettes or over-expression cassettes to generate the desired combination of traits in the plant.

The transformed plants of the invention may be used in a plant breeding program. The goal of plant breeding is to combine, in a single variety or hybrid, various desirable traits. For field crops, these traits may include, for example, resistance to diseases and insects, tolerance to heat, cold, and/or drought, reduced time to crop maturity, greater yield, and better agronomic quality. With mechanical harvesting of many crops, uniformity of plant characteristics such as germination and stand establishment, growth rate, maturity, and plant and ear height, is desirable. Traditional plant breeding is an important tool in developing new and improved commercial crops. This invention encompasses methods for producing a maize plant by crossing a first parent maize plant with a second parent maize plant wherein one or both of the parent maize plants is a transformed plant, as described herein.

Plant breeding techniques known in the art and used in a maize plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids, and transformation. Often combinations of these techniques are used.

The development of maize hybrids in a maize plant breeding program requires, in general, the development of homozygous inbred lines, the crossing of these lines, and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular maize plant using transformation techniques, could be moved into another line using traditional breeding techniques that are well known in the plant breeding arts. For example, a backcrossing approach is commonly used to move a transgene from a transformed maize plant to an elite inbred line, and the resulting progeny would then comprise the transgene(s). Also, if an inbred line was used for the transformation then the transgenic plants could be crossed to a different inbred in order to produce a transgenic hybrid maize plant. As used herein, “crossing” can refer to a simple X by Y cross, or the process of backcrossing, depending on the context.

The development of a maize hybrid in a maize plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly uniform; and (3) crossing the selected inbred lines with different inbred lines to produce the hybrids. During the inbreeding process in maize, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Transgenic plants of the present invention may be used to produce a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B)×(C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

The following examples are offered by way of illustration and not by way of limitation.

### EXAMPLES

### Example 1

### Expression of Transgenes in Monocot Cells

A plasmid vector is constructed comprising a polynucleotide encoding the full-length polypeptide of SEQ ID NO: 13, 14, 15, 16, 17, 18, 19, 20, 21, 22, 23, 24, 25, 26, 27, 28 or 29, operably linked to a heterologous promoter, such as a constitutive promoter or a stress-responsive promoter, for example rab17, rd29A, rip2, mlip15, or ryeCBF31. This construct can then be introduced into maize cells by the following procedure.

Immature maize embryos are dissected from developing caryopses. The embryos are isolated 10 to 11 days after pollination when they are 1.0 to 1.5 mm long. The embryos are then placed with the axis-side facing down and in contact with agarose-solidified N6 medium (Chu, et al., (1975) Sci. Sin. Peking 18:659-668). The embryos are kept in the dark at 27° C. Friable embryogenic callus, consisting of undifferentiated masses of cells with somatic proembryoids and embryoids borne on suspensor structures, proliferates from the scutellum of these immature embryos. The embryogenic callus isolated from the primary explant can be cultured on N6 medium and sub-cultured on this medium every 2 to 3 weeks.

The plasmid p35S/Ac (Hoechst Ag, Frankfurt, Germany) or equivalent may be used in transformation experiments in order to provide for a selectable marker. This plasmid contains the Pat gene (see, European Patent Publication Number 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell, et al., (1985) Nature 313:810-812) and the 3′ region of the nopaline synthase gene from the T-DNA of the Ti plasmid of Agrobacterium tumefaciens.

The particle bombardment method (Klein, et al., (1987) Nature 327:70-73) may be used to transfer genes to the callus culture cells. According to this method, gold particles (1 μm in diameter) are coated with DNA using the following technique. Ten μg of plasmid DNA are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the corn tissue with a Biolistic PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covers a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of actively growing callus about 1 cm in diameter can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm, et al., (1990) Bio/Technology 8:833-839).

### Example 2

### Expression of Transgenes in Dicot Cells

Soybean embryos are bombarded with a plasmid comprising a CBF polynucleotide operably linked to a promoter, as follows. To induce somatic embryos, cotyledons of 3-5 mm in length are dissected from surface-sterilized, immature seeds of the soybean cultivar A2872, then cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with fluorescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A DuPont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188), and the 3′ region of the nopaline synthase gene from the T-DNA of the Ti plasmid of Agrobacterium tumefaciens. The expression cassette comprising the sequence of interest operably linked to a promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 3

### Identification of the Gene from a Computer Homology Search

Gene identities can be determined by conducting BLAST (Basic Local Alignment Search Tool; Altschul, et al., (1993) J. Mol. Biol. 215:403-410; see also, information available from NCBI (National Center for Biotechnology Information, US National Library of Medicine, 8600 Rockville Pike, Bethesda, Md. 20894)) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences are analyzed for similarity to all publicly available DNA sequences contained in the “nr” database using the BLASTN program. The DNA sequences are translated in all reading frames and compared for similarity to all publicly available protein sequences contained in the “nr” database using the BLASTX program (Gish and States, (1993) Nature Genetics 3:266-272) provided by the NCBI. In some cases, the sequencing data from two or more clones containing overlapping segments of DNA are used to construct contiguous DNA sequences.

Sequence alignments and percent identity calculations can be performed using software such as GAP, BestFit, PileUp or Pretty, available as part of the GCG® Wisconsin Package™ from Accelrys, Inc., San Diego, Calif. Default parameters for pairwise alignments of polynucleotide sequences using GAP and BestFit are Gap Creation Penalty=50, Gap Extension Penalty=3; nwsgapdna.cmp is the scoring matrix. Default parameters for pairwise alignments for polypeptide sequences using GAP and BestFit are Gap Creation Penalty=8, Gap Extension Penalty=2; BLOSUM62 is the scoring matrix. There is no penalty for gaps at ends of polynucleotide or polypeptide alignments.

Default parameters for polynucleotide sequence comparison using PileUp and Pretty are: Gap Creation Penalty=5, Gap Extension Penalty=1. Default parameters for polypeptide sequence comparison using PileUp or Pretty are Gap Creation Penalty=8, Gap Extension Penalty=2; BLOSUM62 is the scoring matrix.

Sequence alignments can also be accomplished with the Megalign program of the LASERGENE bioinformatics computing suite (DNASTAR Inc., Madison, Wis.). Multiple alignment of the sequences can be performed using the Clustal method of alignment (Higgins and Sharp, (1989) CABIOS. 5:151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

Other pairwise comparison tools are also available and known to those of skill in the art.

### Example 4

### Standard Agro Transformation Protocol

For Agrobacterium-mediated transformation of maize, the method of Zhao is employed (U.S. Pat. No. 5,981,840, and PCT Patent Publication Number WO98/32326, the contents of which are hereby incorporated by reference). Briefly, immature embryos are isolated from maize and the embryos immersed in an Agrobacterium suspension, where the bacteria are capable of transferring the gene of interest to at least one cell of at least one of the immature embryos (step 1: the infection step). The embryos are then co-cultured for a time with the Agrobacterium on solid medium (step 2: the co-cultivation step). During the co-cultivation step infected embryos are cultured at 20° C. for 3 days, and then at 26° C. for 4 days. Following this co-cultivation period an optional “resting” step is contemplated in which the embryos are incubated in the presence of at least one antibiotic known to inhibit the growth of Agrobacterium, without the addition of a selective agent for plant transformants (step 3: resting step). Transient expression based on a color marker can be monitored during the co-cultivation and the resting steps. Next, inoculated embryos are cultured on medium containing a selective agent and growing transformed callus is recovered (step 4: the selection step). Finally, calli grown on selective medium are cultured on solid medium to regenerate transformed plants (step 5: the regeneration step).

### Example 5

### Identification and Phylogeny of Multiple Maize CBF Polypeptides

As described in Example 3, bioinformatics search tools can be used to identify polynucleotides or polypeptides with common sequences or sequence elements. Using ZmCBF1 and ZmCBF2 sequences (SEQ ID NOS: 1-4), such searches of the TIGR GSS assembly 4.0 were conducted. Seventeen maize CBF or CBF-like sequences were identified in this way.

Maize CBF protein sequences were aligned with Arabidopsis and rye CBF sequences. From the alignment, 1000 half-delete jackknife permuted datasets were generated and used to produce 1000 neighbor-joining phylogenetic trees. The consensus tree from among these was then run through the Maximum-Likelihood program of Phylip to produce a tree with branch lengths scaled to amino acid substitution distance. Based on this tree, all of the corn sequences are in a separate lade from the Arabidopsis sequences. However, the corn sequence lade forms a 100% supported grouping with the Arabidopsis CBF and At5g51990 lade. This grouping suggests that there are four Arabidopsis CBF type proteins and ten corn CBF type proteins.

### Example 6

### Expression Analysis of ZmCBF Genes

For genes ZmCBF3, CBF4 through CBF9, and CBF11, expression profiling was conducted using massively parallel sequencing technology (MPSS, Illumina®, Hayward, Calif.; formerly Solexa). No appropriate signature tags were available for ZmCBF1, ZmCBF2, and CBF 10.

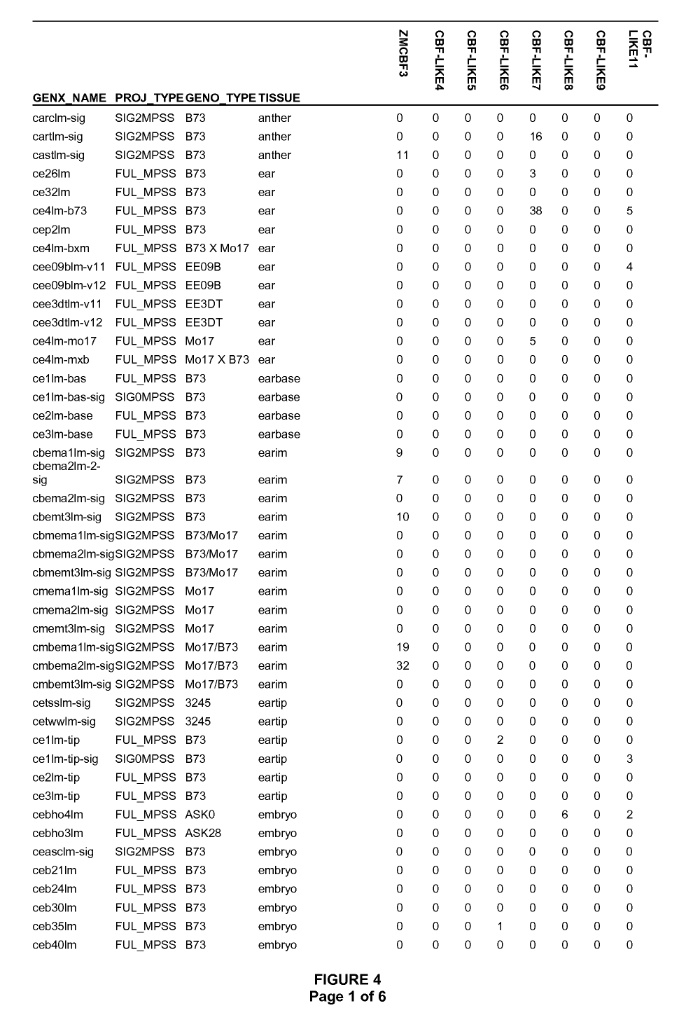

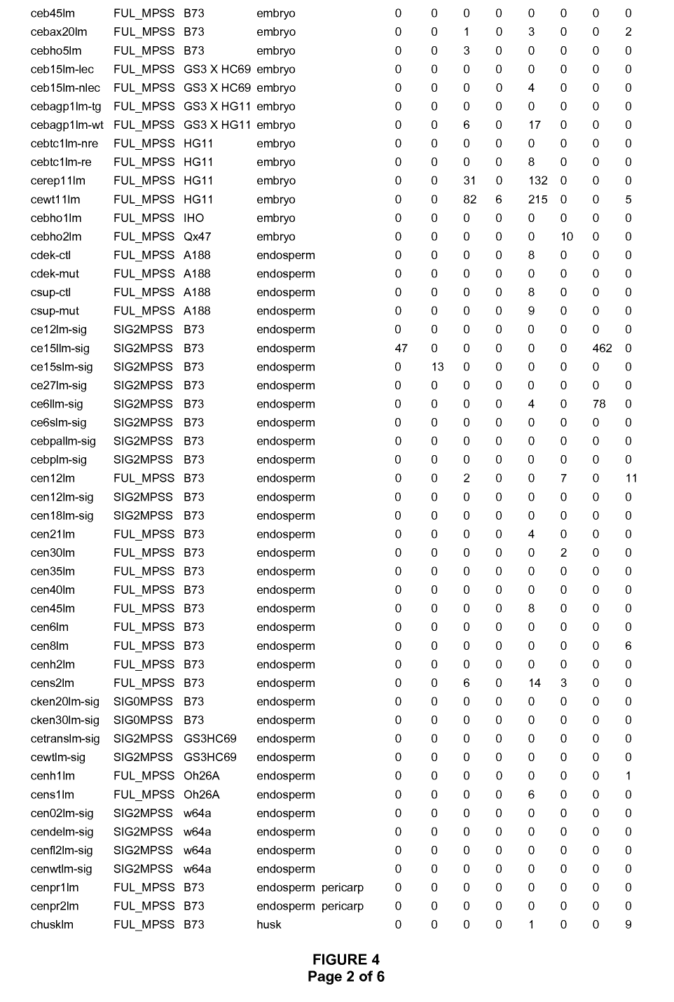

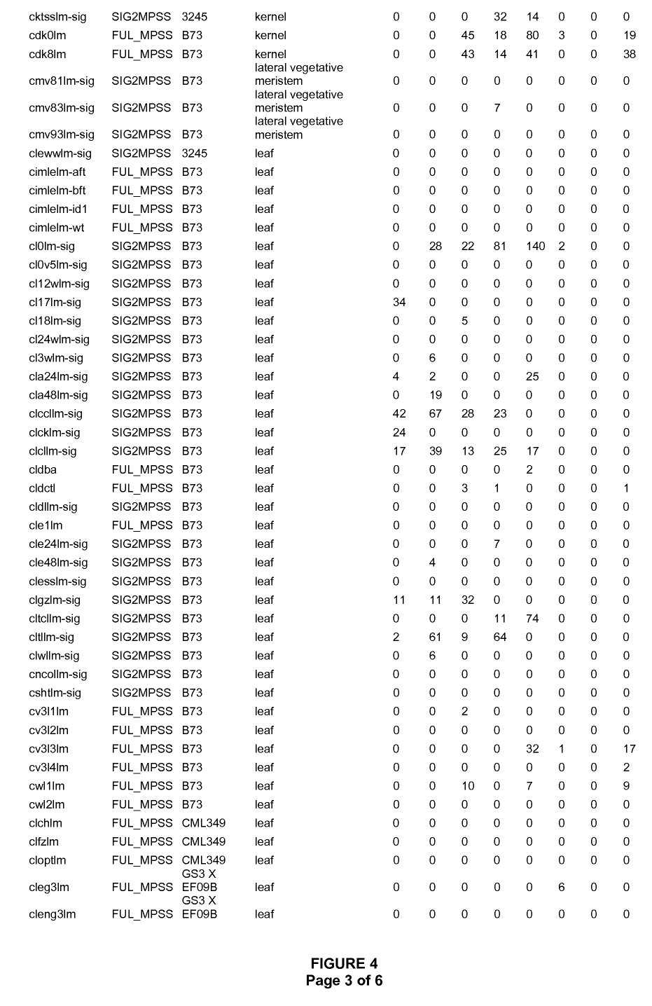

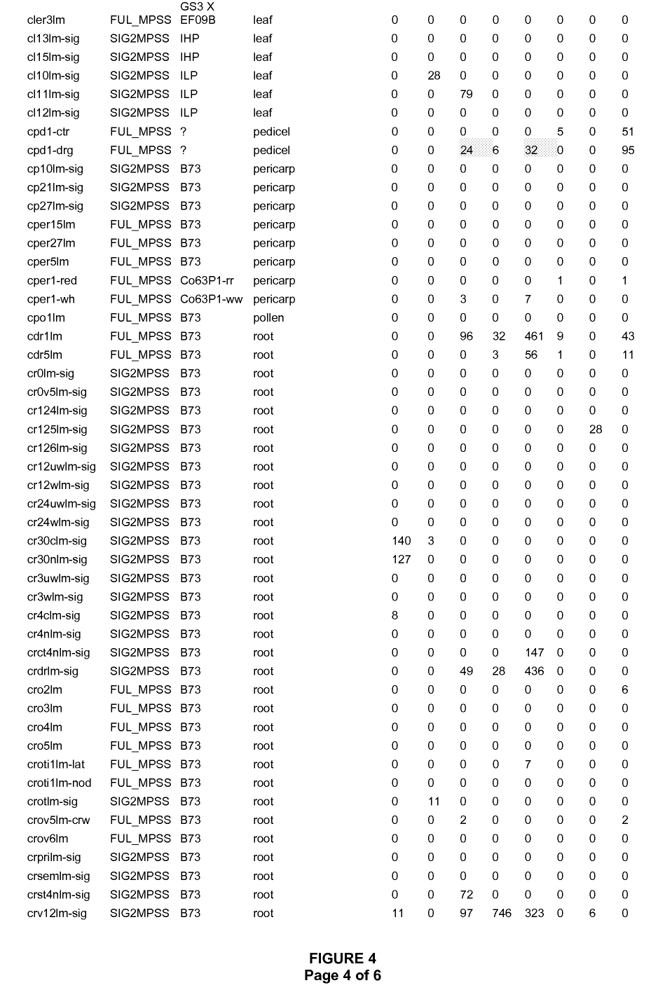

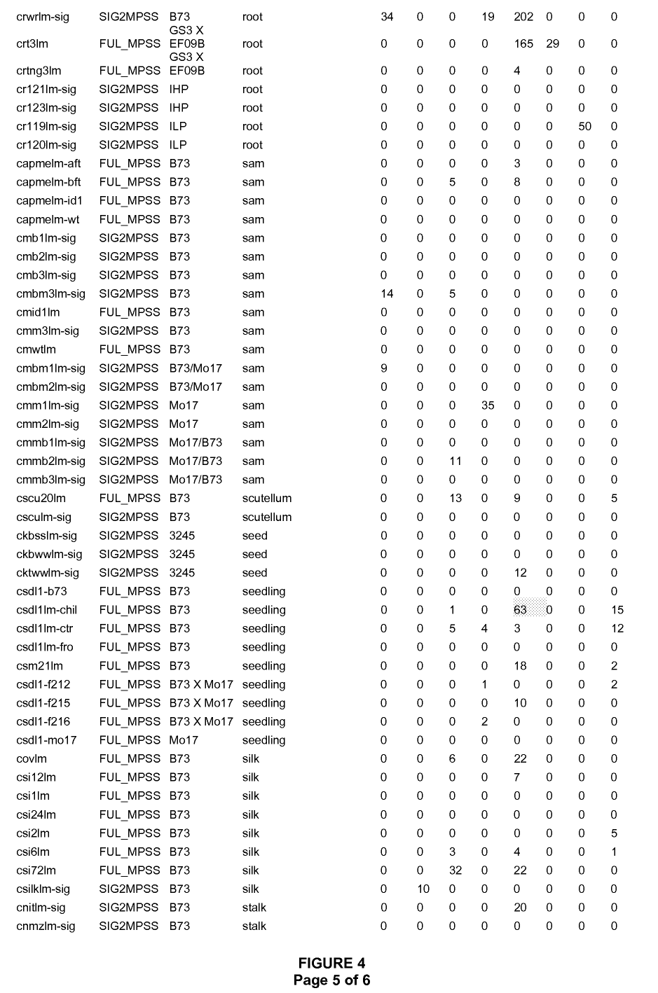

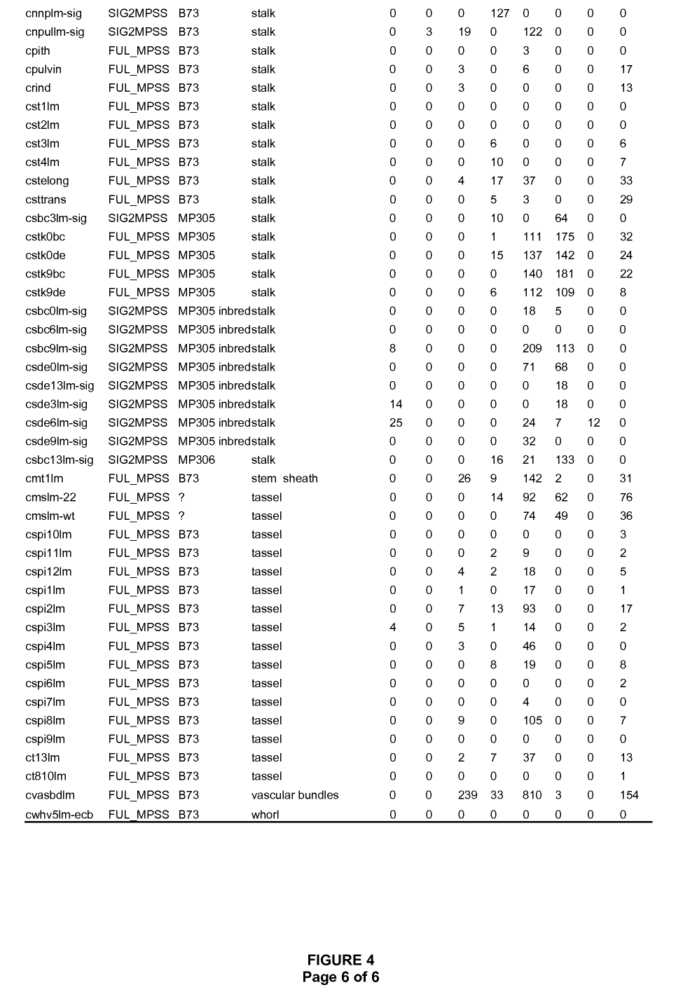

Results are shown in FIG. 4. CBF-like7 is specifically higher in expression in the chilled seedling versus the control; see Page 5 of FIG. 4, csdl1lm-chil versus csdl1lm-ctr. CBF5 and CBF7 are specifically higher in the drought stressed pedicels versus the controls; see Page 4 of FIG. 4, cpd1-drg v. cpd1-ctr.

### Example 7

### ZmCBF12 Expression Data

Analysis of proprietary tissue libraries indicated that ZmCBF12 is expressed in all tissues, namely, vegetative, reproductive, and root, and it was found to be induced by biotic and abiotic stresses. The expression of this gene was highest at 550 ppm in maize whole kernels as reported in the proprietary MPSS libraries. Its expression was four-fold higher in drought-stressed maize pedicels relative to control, almost three-fold higher in ABA-treated leaves and cytokinin-treated leaf discs relative to control, and two-fold higher in seedling tissues that were recovering from freeze-treatment relative to control seedlings at optimum temperatures. This indicates potential significance of this gene in stress tolerance.

The above examples are provided to illustrate the invention but not to limit its scope. Other variants of the invention will be readily apparent to one of ordinary skill in the art and are encompassed by the appended claims.

All publications and patent applications cited in the specification are indicative of the level of skill of those in the art to which this invention pertains. All publications, patents, patent applications, and computer programs cited herein are incorporated by reference to the same extent as if specifically and individually indicated to be incorporated by reference.

That which is claimed is:

1. An isolated polynucleotide encoding a transcription factor which is involved in modulation of gene expression in response to abiotic stress and which comprises the full length amino acid sequence of SEQ ID NO: 13, 14, 16, 25 or 27.

2. The isolated polynucleotide of claim 1 wherein said abiotic stress is due to low temperature or dehydration.

3. An expression cassette comprising an isolated polynucleotide of claim 1 and a promoter sequence operably linked to said polynucleotide, wherein said promoter initiates transcription of said linked polynucleotide in a plant transformed with said expression cassette.

4. The expression cassette of claim 3 wherein said operably linked promoter drives expression in a stress-responsive or tissue-preferred manner.

5. A plant, or a part thereof, stably transformed with an expression cassette of claim 3.

6. The plant part of claim 5, wherein the plant part is selected from the group consisting of: cell, protoplast, cell tissue culture, callus, cell clump, embryo, pollen, ovule, seed, flower, kernel, ear, cob, leaf, husk, stalk, root, root tip, anther and silk.

7. A transgenic seed of the plant of claim 5.

8. The plant of claim 5, wherein said plant is a monocot.

9. The plant of claim 8, wherein said monocot is maize, barley, wheat, oat, rye, sorghum or rice.

10. The plant of claim 5, wherein said plant is a dicot.

11. The plant of claim 10, wherein said dicot is soybean, alfalfa, safflower, tobacco, sunflower, cotton or canola.

12. A method for increasing plant tolerance to abiotic stress, comprising transforming a plant with a transformation vector comprising an isolated polynucleotide encoding a transcription factor which is involved in modulation of gene expression and is at least 90% identical to the full length of SEQ ID NO: 13, 14, 16, 25 or 27, as determined by GAP analysis under default parameters.

13. The method of claim 12, wherein said abiotic stress is due to low temperature or dehydration.

14. The method of claim 12, wherein said polynucleotide is operably linked to a promoter which drives expression in a stress-responsive or tissue-preferred manner.

15. An isolated polynucleotide encoding a transcription factor which is involved in modulation of gene expression in response to abiotic stress and which is at least 85% identical to the full length of SEQ ID NO: 13, 14, 16, 25 or 27, as determined by GAP analysis under default parameters.

16. An isolated polynucleotide of SEQ ID NO: 30, 37, 39, 43 or 44.

17. An isolated polynucleotide at least 85% identical to the full length of the polynucleotide of claim 16 encodes a transcription factor involved in modulation of gene expression in response to abiotic stress.
